(19) United States
(12) Patent Application Publication
(10) Pub. No.: US 2013/0005028 A1
(43) Pub. Date: Jan. 3, 2013

(54) Activation of RIG-I Pathway

(76) Inventors: Larry R. Smith, San Diego, CA (US); Catherine K. Yang, San Diego, CA (US)

(21) Appl. No.: 12/520,074

(22) PCT Filed: Dec. 21, 2007
(86) PCT No.: PCT/US07/88620

§ 371 Date: Mar. 4, 2010

Related U.S. Application Data
(60) Provisional application No. 60/871,336, filed on Dec. 21, 2006.

(51) Int. Cl. C12N 15/63

(52) U.S. Cl. 435/320.1

(57) ABSTRACT

The disclosed invention relates to nucleic acid molecules and compositions thereof for stimulating or enhancing an immune response in a subject. Methods for the use of the molecules and compositions, including administration of the molecules or compositions to a subject to facilitate an immune response to a vector encoded antigen are also described.

### CROSS REFERENCE TO OTHER APPLICATION

This application claims benefit of priority to U.S. Provisional application 60/871,336, filed Dec. 21, 2006, the disclosure of which is fully incorporated herein by reference.

### FIELD OF THE DISCLOSURE

This disclosure relates to the fields of immunology and the generation of an immune response in a subject to a nucleic acid encoded antigen. Nucleic acid molecules and compositions thereof for stimulating or enhancing an immune response in a subject are disclosed. Methods for the administration of the molecules or compositions to facilitate an immune response to a vector encoded antigen are also described.

### BACKGROUND OF THE DISCLOSURE

Innate immune responses can be triggered by a variety of pathogen-associated molecular patterns (PAMPs) which directly provide defense mechanisms such as antiviral activity and indirectly provide signals for enhancing adaptive immune responses. Toll-like receptors (TLR) are a family of receptors which recognizes a wide variety of PAMPs including LPS, dsDNA (double-stranded DNA), dsRNA (double-stranded RNA), ssRNA (single-stranded RNA), flagellin, and other pathogen-derived signature structures. The stimulation of PAMP-responsive signaling pathways by various TLR agonists has been shown to be critical for adaptive Th1 responses (Schnare, Nat Imm 2001) as well as for overcoming the suppressive effects of CD4+CD25+ regulatory T cells (Pasare, Science 2003).

Adaptor proteins and signaling pathways have been defined for many of the TLR (Akira Nat Rev, 2004). For example, TLRs that recognize either dsRNA (TLR 3) or ssRNA (TLR 7/TLR 8) signal through intracellular adaptor proteins such as TRIF and MyD88, respectively. The subsequent downstream transcription factors that are activated by TLR3 are IRF 3 and NFkB while TLR 7/8 activates IRF7. All of these transcription factors can in turn activate pro-inflammatory genes such as the type I IFN and a variety of other cytokines. While such RNA-triggered TLR signaling pathways have been shown to augment adaptive immune responses, other pathways independent of TLR, but dependent on activation of the same downstream transcription factors, have been reported to also enhance immune responses.

The type I IFN family in humans and mice include a single IFN-β gene and 14 IFN-α genes as well as several other IFN genes (Hertzog, Trends Imm, 2003). Nearly every cell type, including fibroblasts and epithelial cells, may produce type I IFNs in contrast to the strict production of, and responsiveness to, the type II IFN-γ by some leukocytes. Similarly, nearly every cell type expresses the IFN alpha/beta receptor while only select cells express the IFN-γ receptor. While type I IFNs are best known for direct antiviral effects, recent studies have provided evidence that they can enhance several components of adaptive immune responses as follows: stimulation of DC maturation (Luft, J. Imm. 1998); stimulation of proliferation of memory CD8+ cells by inducing IL-15 (Tough, Immunity, 1998); prevention of activation-induced T cell death (Marrack JEM 1999); enhancement of antibody responses (Le Bon Immunity 2001); activation of STAT4-dependent IFN-γ production (Nguyen Science 2002); stimulation of CD4-independent cross-priming of CD8+ T cells (Le Bon Nat Imm 2003); provision of signal 3 to CD8+ T cells to stimulate expansion and differentiation (Curtsinger, J. Imm., 2005); and type I IFN combined with influenza antigens induced enhanced immune responses in mice (Proietti, J Imm., 2002).

Type I IFNs have rarely been tested for adjuvant activity in plasmid DNA (pDNA) vaccine studies. In a 2002 review of cytokines and chemokines used as genetic adjuvants for pDNA vaccines (Egan, Clin. App Imm Rev, 2002), 165 published studies were reported using 30 different molecules. Of these only one 1 study tested the co-expression of a type I interferon (Tuting, Gene Therapy 1999).

Citation of the documents herein is not intended as an admission that any document is pertinent prior art. All statements as to the date or representation as to the contents of the documents is based on available information and does not constitute any admission as to the correctness of the dates or contents of these documents.

### SUMMARY OF THE DISCLOSURE

This disclosure relates to the enhancement of a subject's immune response, such as to an antigen of interest encoded by a nucleic acid vector. The enhancement is based in part on the ability to activate the retinoic acid-inducible gene I (RIG-I) pathway and so stimulate or activate adaptive immune responses in a cell, or a subject containing the cell, to an antigen expressed via the vector. In many cases, activation of the pathway leads to an increase in type I interferon production and/or the release of pro-inflammatory cytokines in the cell or a subject containing the cell. The coordinated activation of adaptive immunity with antigen expression in the same cell potentiates antigen-specific adaptive immune responses in the subject.

In a first aspect, the disclosure includes a nucleic acid molecule comprising, and capable of expressing, a first coding region encoding a RIG-I pathway agonist and a second coding region encoding an antigen. In some embodiments, the RIG-I pathway agonist is a RIG-I agonist, or agonist of RIG-I activity in the pathway. Non-limiting examples of RIG-I activity include intracellular interactions with mitochondrial antiviral signaling (MAVS) protein which lead to increased levels of interferon-β (IFN-β) or increased IFN-β production. In other embodiments, the RIG-I pathway agonist is an agonist of MAVS activity which leads to increased IFN-β levels. Alternatively, and without being bound by theory but offered to improve the understanding of the disclosed subject matter, a RIG-I agonist or an MAVS agonist of the disclosure directly or indirectly activates the NFκB and/or interferon regulatory factor 3 (IRF3) signaling pathways.

Agonists of the RIG-I pathway include molecules that are polypeptide or nucleic acid in structure. Non-limiting examples include polypeptides, such as fragments of the RIG-I protein, which interact with MAVS to result in increased levels of intracellular IFN-β production or the activation of the NFκB and/or interferon regulatory factor 3 (IRF3) signaling pathways. Other examples of a polypeptide agonist include the MAVS protein, or fragments thereof, which result in the same cellular activity. In some cases, the agonist activity is provided by overexpression of MAVS or a fragment thereof, such as by use of a strong promoter or enhancers to increase intracellular expression. In other embodiments, a RIG-I agonist may be a DNA or RNA molecule, such as a double-stranded DNA or RNA (dsRNA) or single-stranded DNA or RNA (ssRNA) molecule. In some cases, the RNA molecule comprises a 5′ triphosphate moiety.

The antigen encoded by the nucleic acid molecule may be any of interest and to which an immune response in a subject is desirable. In some cases, the antigen is that of a pathogen such that the immune response thereto results in an immunized state, in a subject, against the antigen or the pathogen. Embodiments of a pathogen include a virus or other microorganism. In other embodiments, the antigen is that of a tumor or other cancer cell. An antigen of the disclosure is, of course, capable of inducing an immune response upon expression in the cell or subject.

The first aspect of the disclosure includes a DNA vector comprising the first and second coding regions, which may be in any suitable orientation such that intracellular expression of the encoded agonist and antigen may occur. In some embodiments, the vector is a closed circular molecule, or plasmid. In alternative embodiments, the vector is linear in structure. In further embodiments, the vector may be a viral vector comprising the coding regions. The coding regions of the vector are fused in some embodiments such that a single RNA transcript is produced with sequences complementary to both coding regions. A vector comprising a fused arrangement of coding regions includes a construct wherein the regions are in a tandem (head to tail) orientation. In some embodiments, the tandem orientation begins with the 5′ end of the coding region for the agonist followed by the 5′ end of the coding region for the antigen. A fused arrangement allows for the use of a single promoter or other regulatory element(s) in the vector to direct or regulate the expression of the coding regions.

Alternatively, the vector comprises separate coding regions, present as part of two separate transcription units, such that their expression result in the production of individual RNA transcripts with sequences complementary to each of the first and second coding regions. The arrangement of coding regions, or transcription units, may thus be tandem (head to tail) or inverted (head to head or tail to tail) in orientation. In some embodiments, a vector comprises a tandem orientation with each coding region under the control of its own promoter or other regulatory element(s) in the vector. The expression of each coding region is thus under the direction or regulation of its own promoter or regulatory element(s) region. In some alternative embodiments, the two transcription units utilize non-promoter regulatory elements in common. In other embodiments, the vector comprises an inverted orientation wherein transcription of the two coding regions result in transcription complexes that move away from each other (divergent orientation) or wherein the transcription complexes move toward each other (convergent orientation).

A promoter operatively linked to a coding region in a vector of the disclosure maybe recognized by any suitable cellular RNA polymerase, including the RNA polymerase I (Pol I), RNA polymerase II (Pol II), and RNA polymerase III (Pol III) activities of eukaryotic cells. Embodiments of the disclosure include a vector with a combination of a promoter operably linked to a coding region where the combination is found in a naturally occurring nucleic acid molecule. The promoter may be considered endogenous to the coding region, and vice versa. In other embodiments, a vector comprises a promoter operatively linked to a coding region wherein the two are not found in a naturally occurring nucleic acid molecule. The promoter may thus be considered exogenous or heterologous to the coding region.

The first aspect of the disclosure further includes a composition containing the nucleic acid molecule. In some embodiments, the composition comprises a pharmaceutically acceptable excipient or carrier for delivery of the nucleic acid molecule to a cell or a subject.

In a second aspect, the disclosure includes a composition comprising a first nucleic acid molecule comprising a coding region encoding a RIG-I pathway agonist and a second nucleic acid molecule comprising a coding region encoding an antigen. Such an arrangement presents each coding region via a separate transcription unit on a separate nucleic acid molecule as opposed to coding regions on a single molecule. The nature of the encoded RIG-I pathway agonist and antigen is as described herein. The composition may also be considered to comprise two vectors, with one or both being a viral vector, with one comprising the first coding region and a second comprising the second coding region. In some embodiments, the composition comprises a pharmaceutically acceptable excipient or carrier for delivery of one or both of the nucleic acid molecules to a cell or a subject.

Of course the disclosure also includes each of these first and second molecules, or vectors, separate from the other. Non-limiting examples include each vector during its preparation or production as well as in a method of administering the two vectors separately rather than via a single composition. Independently, each of the molecules, or vectors, may also be closed circular (plasmid) or linear in structure.

Given the separation of the coding regions on two vectors, each coding region is operatively linked to its own promoter or regulatory region which controls the expression of the encoded agonist or antigen. The range of promoters operatively linked to the coding region in each vector include RNA Pol I, Pol II, or Pol III promoters as described herein. In some embodiments, the first coding region is operably linked to an RNA Pol III, or RNA Pol I, promoter capable of directing or regulating expression of the agonist. This may be optionally combined with the second coding region being operably linked to an RNA Pol II promoter capable of directing or regulating expression of the antigen.

A third aspect of the disclosure includes a method of inducing or producing an immune response, in a subject, to an antigen. The method may comprise administering to the subject, a first coding region encoding a RIG-I pathway agonist, and a second coding region encoding an antigen, under conditions wherein the first and second coding regions are expressed in said subject to induce or produce an immune response. In some embodiments, the first and second coding regions are contained in a single nucleic acid molecule, such as a DNA vector, as described herein. In other embodiments, the two coding regions are contained on separate vectors which may be optionally administered via a single composition containing them. Alternatively, the two separate vectors may be separately administered to a subject.

In a fourth aspect, the disclosure includes a nucleic acid molecule comprising a coding region encoding an RNA agonist of the RIG-I pathway. In some embodiments, the RNA agonist is a RIG-I agonist as described herein. In other embodiments, the coding region is operatively linked to an RNA Pol III promoter, such as a Pol III promoter which produces RNA transcripts comprising a 5′ triphosphate moiety. In some cases, the Pol III promoter is heterologous relative to the coding region such that the two are not found in a naturally occurring nucleic acid molecule. In other cases, the promoter and coding region are normally found operatively linked in a naturally occurring nucleic acid molecule. One non-limiting example is in the case of a promoter and coding region combination, as found in a naturally occurring viral sequence, comprising a viral promoter operatively linked to a coding region encoding an viral RNA agonist. The nucleic acid molecule may be isolated or purified, and in the case of a recombinantly produced molecule, the isolation or purification may be after its preparation by recombinant methods. The vector is optionally in a closed circle (plasmid) or linear configuration.

The nucleic acid molecule, or a composition comprising it, may be used in a method of inducing, increasing, enhancing, stimulating, or producing an immune response in a subject. The method may comprise introducing a DNA molecule encoding, or an RNA molecule that functions as, an RNA agonist of the RIG-I pathway, into a cell of a subject under conditions wherein the nucleic acid is expressed in said cell. Optionally, the method further comprises introduction of a coding region encoding, and capable of expressing, an antigen. Embodiments include, but are not limited to, methods wherein the coding region encoding an antigen is present on the DNA molecule, on the RNA molecule that functions as an agonist, or on a second nucleic acid molecule as described herein. The presence of both the RNA agonist and antigen in the cell results in the coordinated activation of an adaptive immune response to potentiate antigen-specific responses in the subject. RIG-I pathway agonist polypeptides may be used in place of, or in addition to, the DNA or RNA molecules for the methods of the disclosure.

In a further aspect, the disclosure includes a method of increasing IFN-β, or cytokine or chemokine, levels or production in a cell or subject containing the cell. The method may optionally be used to increase type I IFN levels in the subject. In some embodiments, the method comprises introducing an isolated nucleic acid molecule comprising a coding region encoding an RNA agonist of the RIG-I pathway as described herein. In some embodiments, the method may comprise an isolated DNA vector encoding an RNA agonist of the RIG-I pathway introduced into said cell under conditions wherein the nucleic acid is expressed in said cell. In other embodiments, the method further comprises introducing into the cell a coding region encoding, and capable of expressing, an antigen as described herein. In some cases, the increased production is of a pro-inflammatory cytokine, with IL-6, IP-10, IL-1, IL-12, and RANTES as non-limiting examples. In further embodiments, increased IFN-□ levels or production in a subject may advantageously result in the activation of dendritic cells (DCs), enhance antibody responses, and/or enhance CD8+ responses induced by cross priming.

The disclosure also includes additional embodiments of the above methods that further comprise contacting a cell expressing an agonist of the RIG-I pathway with interferon-□ (IFN-□) and/or tumor necrosis factor alpha (TNF-□). In embodiments of these methods wherein the cell is within a subject, the IFN-□ and/or TNF-□ may be administered to the subject separately, or together with, the nucleic acid molecule encoding the agonist. The methods may be used to further stimulate, or accelerate, an increase in type I IFN levels or production in a subject containing the cell.

The details of additional embodiments are set forth in the accompanying drawings and the description below. Other features, objects, and advantages of the embodiments will be apparent from the drawings and detailed description, and from the claims.

### BRIEF DESCRIPTION OF THE DRAWINGS

FIG. 1. Activation of IFN-β and IP-10 responses in vitro by RNA or ΔRIG-I encoding pDNA. VM92 (A, C) and L929 (B, D) cells were transfected in 24-well plates with 100 or 250 ng of the indicated RNA or 250 ng of plasmid DNA (A). 48 h after transfection, cell culture supernatants were collected and assayed for (A, B & C) IFN-β or (D) IP-10 by ELISA assays.

FIG. 2 Enhancement of cytokine levels in mouse muscle and serum in vivo. Mice were intramuscularly injected with VR10551 vector backbone, VR9033 (ΔRIG-I) pDNA or poly I:C in PBS (100 μg/animal, 50 μl/site). 6 h post-injection, sera and muscle tissues were collected and assayed for muscle (A) IP-10 or (B) IL-1β and (C) serum IP-10 levels by ELISA assays. n=8 muscles for each group. *: p<0.05 using a Student T-test.

FIG. 3 RNA enhancement of cytokine levels in mouse muscle. Mice were intramuscularly injected with PBS (−) or ML RNA in PBS (+) (100 μg/animal, 50 μl/site). 6 h post-injection, muscle tissues were collected and assayed for the indicated cytokine levels. n=4 muscles for each group. p<0.05 for each cytokine using a Student T-test.

FIG. 4 ML RNA-induced enhancement of humoral immune responses in vivo. Mice were intramuscularly injected on day 0 and day 21 with TIV (1 μg) and ML RNA (100 μg) in PBS as indicated. Serum samples were collected on day 20 and day 33 and assayed for anti-TIV specific antibodies. n=10 for each group. p<0.05 for with and without ML RNA using Wilcoxon rank sum test on the log transformed titer data.

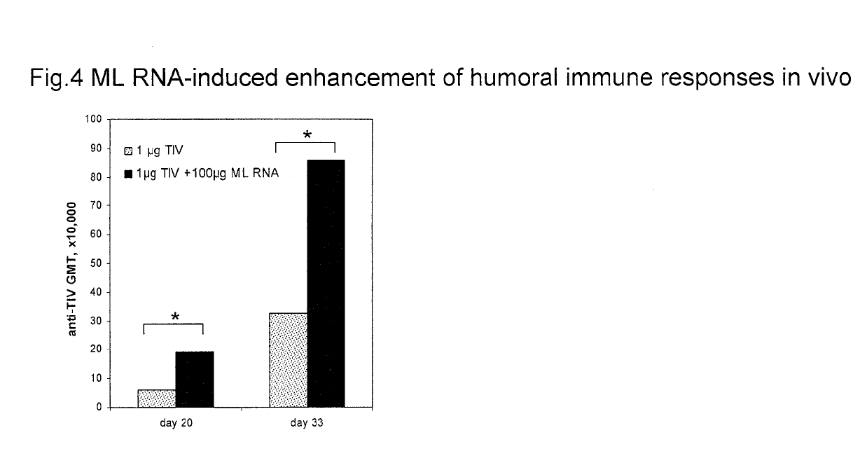

FIG. 5. dsRNA activation by the RIG-I pathway. The helicase domain of RIG-I recognizes dsRNA which facilitates the interaction of RIG-I and MAVS through their CARDS. This enables TBKI and IKKε to phosphorylate IRF3 and NFκB which in turn stimulates IFN-β expression in a cell's nucleus.

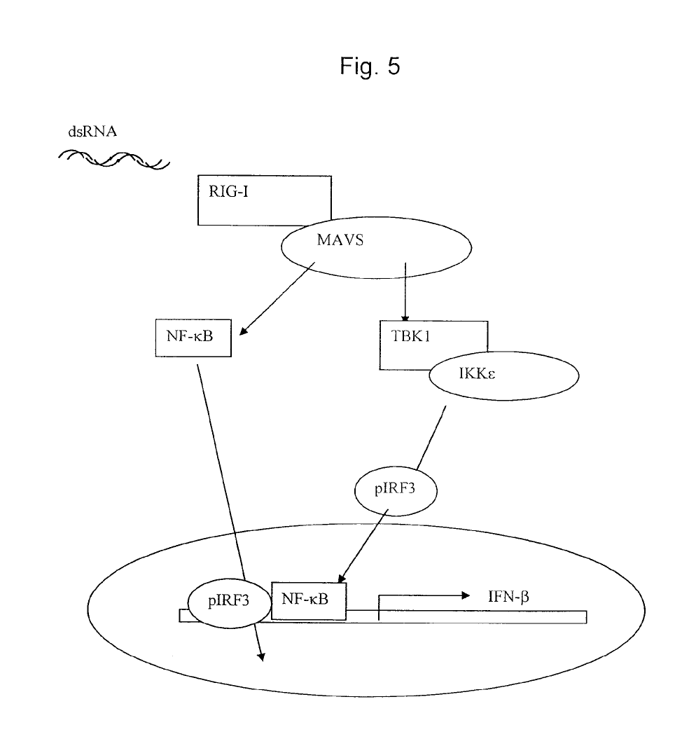

### DETAILED DESCRIPTION AND MODES OF PRACTICING THE DISCLOSURE

### Definitions

As used herein, a “nucleic acid molecule” or “nucleic acid” or “polynucleotide” refers to a polymer of deoxyribonucleotides or ribonucleotides. The polymer may be single-stranded or double-stranded as well as in closed circle (plasmid) or linear form. A polymer may comprise one or more nucleotide analogs that possesses the characteristics of naturally occurring nucleotides in their ability to undergo complementary, basepair based hybridization between single stranded polymers as well as the ability to be transcribed by an RNA polymerase. The range of polymers containing one or more nucleotide analogs includes peptide nucleic acids as well as polymers containing one or more phosphorothioate linkages in the polymeric backbone. In cases of a linear polymer, the one or more phosphorothioates linkages may be at the ends of the polymer to protect the polymer from degradation or increase its stability or half-life in vivo, such as within a cell or a subject of the disclosure.

The terms “nucleic acid molecule” or “nucleic acid” or “polynucleotide” also refer to polymers with the functionality of a deoxyribonucleic acid (DNA or deoxyribopolynucleotide) or ribonucleic acid (RNA or ribopolynucleotide) to hybridize, such as under stringent hybridization conditions, to a sufficiently complementary nucleotide sequence and/or to be expressed as a polypeptide as encoded by the polymer via the genetic code.

A “polypeptide” or “protein” or “peptide” refers to a polymer of amino acid residues linked via peptide bonds. The residues are generally natural as found in a cell which produces the polypeptide. A polypeptide is not limited in size and may be about 5, about 10, about 25, about 50, about 75, about 100, about 150, about 200, about 250, about 300, about 350, about 400, about 450, about 500, about 750, about 1000, about 2000, about 3000, about 4000, about 5000, about 6000, about 7000, about 8000, about 9000, about 10,000 amino acids or longer in size. In some examples of antigens of the disclosure, the antigen is a polypeptide of a size appropriate for intracellular processing and presentation via MHC Class I or Class II molecules. Non-limiting examples include polypeptides of 9 amino acids in length, or a longer length that is processed intracellularly to be presented via MHC Class I or Class II molecules.

A “nucleic acid molecule” or “nucleic acid” or “polynucleotide” molecule of the disclosure may be “isolated” or “purified” which refers to a state of being free of one or more factors or materials which are normally found with, or associated with, the molecule in its naturally occurring environment (such as a cellular environment) or as a result of its method or means of production. So where a molecule occurs in a naturally occurring environment, the molecule may be isolated or purified by direct or indirect human intervention or manipulation to remove it from one or more components normally found with the molecule or one or more contaminants that would interfere with its subsequent use. And where a molecule is produced or prepared by direct or indirect human intervention or manipulation, the molecule may be isolated or purified by removal of one or more components present due to the production or preparation process.

A “nucleic acid molecule” or “nucleic acid” or “polynucleotide” molecule of the disclosure may be “expressed” or “capable of expressing” an encoded product. The term “express” and variations thereof refer to the process wherein a region of the polymeric molecule is used as a template to produce a product, such as a biologically active molecule. The region or portion of a polymer that encodes, and therefore can be expressed as, a biologically active polypeptide or nucleic acid molecule is referred to as the “coding region” or “coding sequence”.

The polymeric molecule may encode a biologically active RNA agonist as described herein or an RNA intermediate that is capable of being translated into a biologically active polypeptide. In the latter case, the polymeric molecule encodes a biologically active polypeptide. Thus a DNA molecule that is operably linked to appropriate regulatory regions, such as a promoter, is a template that expresses, or is capable of expressing, an RNA transcript as the encoded product or a polypeptide as the encoded product via subsequent translation of the RNA transcript. In comparison to a DNA molecule, an RNA molecule is a template that expresses, or is capable of expressing, an encoded polypeptide via translation of the RNA molecule.

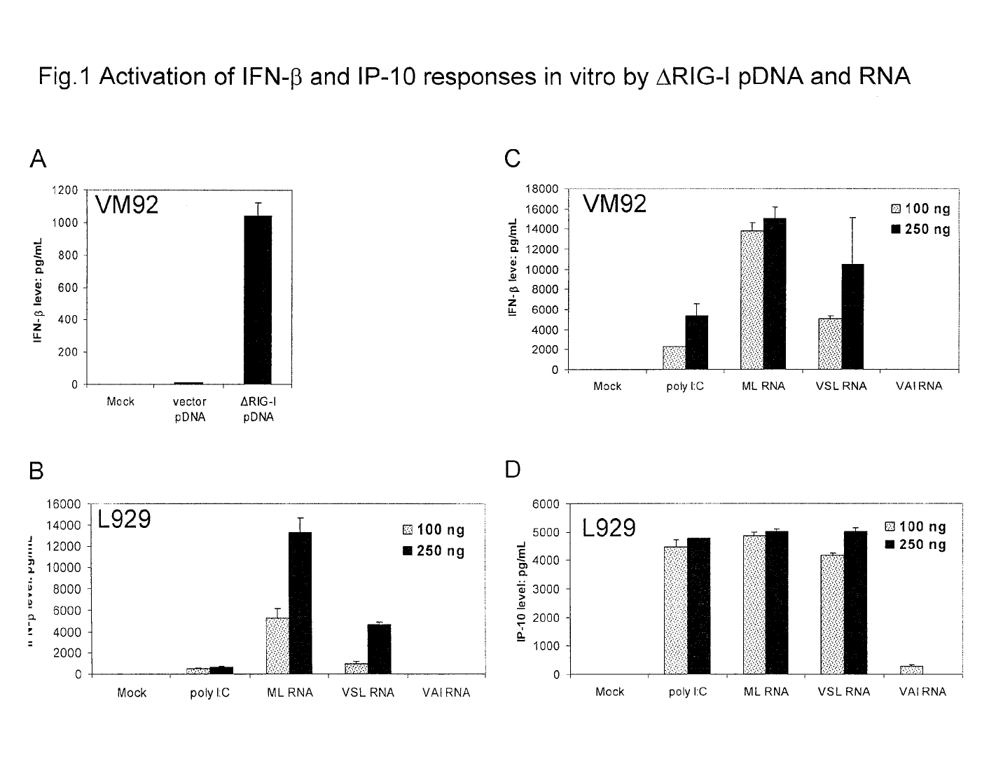

An encoded biologically active molecule includes a RIG-I pathway agonist of the disclosure. “RIG-I pathway” as used herein refers to a reported cellular signaling pathway that includes the RIG-I protein. RIG-I protein has been reported to act as a positive regulator of the type I IFN system in response to dsRNA and signal in a TLR-independent fashion (Yoneyama, Nat. Imm., 2004; see FIG. 1 therein). The RIG-I protein contains two (2) caspase recruitment domains (CARDs), which can interact with CARDs from other proteins, and an RNA helicase domain which binds dsRNA in the cytoplasm (Yoneyama, Nat Imm 2004). RIG-I protein has been reported to be localized in the cytoplasm of a cell. This is in contrast to other PAMP receptors, such as TLR family members, which reside in an endosome or at the plasma membrane of a cell.

The sequence of human RIG-I (SEQ ID NO:1), based upon the mRNA sequence deposited as GenBank accession number AF038963, has been deposited as GenBank AAD19826 (see Sun, Y. W. “RIG-I, a human homolog gene of RNA helicase, is induced by retinoic acid during the differentiation of acute promyelocytic leukemia cell” Thesis (1997) Shanghai Institute of Hematology, Rui-Jin Hospital, Shanghai Second Medical University). Counterpart RIG-I sequences from other mammals and primates have also been deposited. Non-limiting examples include the Macaca mulatta (rhesus monkey) sequence (LOCUS DQ673981), the Mus musculus (house mouse) sequence (LOCUS AY553221), and an exon based Pan troglodytes (chimpanzee) sequence (LOCUS DQ038435).

A reported intermediate protein component of the RIG-I pathway is a CARD-containing mitochondrial antiviral signaling (MAVS) protein, which acts downstream of RIG-I and upstream of other components in the pathway. MAYS expression has been reported to be essential for RNA-triggered activation of NFκB and IRF3 signaling pathways (Seth, Cell 2005). MAVS is identical to recently identified proteins, CARD adaptor inducing interferon beta (Cardif, Meylan, Nature 2005), interferon beta promoter stimulator 1 (IPS-1, Kawai, Nat. Imm. 2005) and virus-induced signaling adaptor (VISA, Xu, Mol Cell, 2005).

The sequence of human MAVS (SEQ ID NO:2), based upon the mRNA sequence deposited as GenBank accession number DQ174270, has been deposited as GenBank AAZ80417 (see Seth et al. “Identification and characterization of MAVS, a mitochondrial antiviral signaling protein that activates NF-κB and IRF 3.” Cell 2005, 122(5):669-82). Counterpart MAVS sequences from other mammals, including a primate, have also been deposited. Non-limiting examples include the Macaca mulatta (rhesus monkey) sequence (LOCUS DQ842019) and the Mus musculus (house mouse) sequence (LOCUS DQ174271).

A RIG-I pathway “agonist” as used herein refers to a molecule that directly or indirectly activates the pathway, and/or directly or indirectly interacts with one or more reported components of the pathway, to increase IFN-β production and/or the activation of the NFκB and/or IRF3 signaling pathways relative to the absence of the agonist. An agonist of the pathway may be a polypeptide or nucleic acid molecule. In some cases, an agonist has a cytoplasmic location, or is directed to a cytoplasmic location, within a cell for more efficient exposure to, or contact with, components of the pathway.

Non-limiting examples of agonists of the disclosure include a polypeptide, RNA molecule, or small organic molecule that interacts with RIG-I (i.e. a “RIG-I agonist” or “agonist of RIG-I”) to activate the pathway, or component thereof (such as MAVS or the NFκB and/or IRF3 signaling pathways) and/or result in IFN-β production. The term “organic molecule” refers to an organic or medicinal molecule or compound which is not solely nucleic acid or polypeptide in structure while the term “small organic molecule” refers to such a molecule or compound that has a molecular weight of less than about 2000 Daltons (or AMU), such as less than about 1000 Daltons or less than about 500 Daltons. Non-limiting examples of an organic molecule include a carbon containing molecule or compound, as well as the members of a library of chemical and/or biological compounds, such as those prepared by chemical synthesis (including combinatorial chemistry methods) or obtained from fungal, bacterial, and/or algal extracts.

Representative RIG-I agonists include, but are not limited to, a polypeptide that competes with RIG-I for interactions with MAVS to activate the pathway subsequent to MAVS (such as activation of the NFκB and/or IRF3 signaling pathways) or to result in IFN-β production; an RNA molecule, either dsRNA or ssRNA, that interacts with RIG-I to result in the same activation of the pathway, or components thereof, or IFN-β production; or a small organic molecule with the above activities of a polypeptide or RNA agonist. In some cases, a polypeptide agonist is a fragment of the RIG-I polypeptide, and an RNA agonist of RIG-I comprises a 5′ triphosphate moiety.

The agonist may also be a small organic molecule or an MAVS polypeptide, or a fragment thereof, that activates the pathway subsequent to MAVS (such as activation of the NFκB and/or IRF3 signaling pathways) or results in IFN-β production. Non-limiting examples include MAVS, which may be overexpressed, such as by use of a strong promoter or enhancer(s) to increase intracellular expression above that normally present in a cell; and a fragment of MAVS that competes with MAVS for interactions with its cellular binding partner(s) to activate the pathway subsequent to MAVS (such as activation of the NFκB and/or IRF3 signaling pathways) or to result in IFN-β production.

As used herein, “antigen” refers to a molecule capable of being encoded and expressed by a nucleic acid molecule. Non-limiting examples include peptides, polypeptides, proteins, and protein domains. An antigen is bound, via one or more epitopes thereof, by an antibody, or a T cell receptor (TCR) when presented via an MHC molecule. Thus an antigen may be monovalent or multivalent (polyvalent) in the epitopes it presents to B- and T-lymphocytes. An antigen is recognized by a subject's immune system, and optionally capable of inducing an immune response, such as a humoral immune response and/or cellular immune response, in the subject as described herein. An antigen may also generate or induce an immune response which comprises increase levels or production of one or more type I IFN, IFN-β, and/or one or more pro-inflammatory cytokines, by a cell or subject in comparison to the absence of the antigen.

An antigen of the disclosure may be expressed in combination with one or more other antigens. Non-limiting examples of antigens include tumor or cancer antigens, viral antigens, bacterial antigens, fungal antigens, microalgal antigens, antigens of pathogenic organisms (pathogens), and antigens derived from infectious disease agents.

Expression of an agonist and/or antigen from a nucleic acid coding region is mediated by one or more operably (or operatively) linked “regulatory elements”. Two sequences, such as a coding sequence and a promoter as a regulatory element, are “operably linked” if induction of promoter function results in the transcription of the coding sequence. Preferably, the nature of the linkage between the two sequences does not (1) result in the introduction of a frame-shift mutation, (2) interfere with the ability of the regulatory sequences to direct expression of the linked coding region, or (3) interfere with the ability of the sequence to be transcribed. Some promoters and regulatory elements are operably linked to a coding region without being located 5′ to the region.

A “regulatory element” refers to nucleotide sequences that induce or control transcription of an operably linked nucleic acid template or coding region. Non-limiting examples of regulatory elements include initiation signals, enhancers, and promoters (see Goeddel; Meth Enz 185 (1990) for additional examples and discussion). A promoter sequence (or other regulatory element) is used as described herein to induce or control expression of a coding region in a cell in which expression is desired or intended. Non-limiting examples of promoters include tissue-specific promoters (which regulate expression of an operably linked DNA sequence in specific cells of a tissue of a subject); constitutive promoters (which remain active in a cell under most conditions, e.g. temperature or oxygen level, to which the cell is exposed); inducible promoters (which is under environmental or other regulation such as the presence of an exogenous agent, e.g. antibiotic, or exposure to a specific condition, e.g. temperature or reduced oxygen levels); or leaky promoters (which regulate expression primarily in certain tissue(s) or cell type(s), but allow expression to occur in other tissues or cells. An “exogenous” agent refers to a molecule or compound that is not normally or naturally present in or with a cell or subject.

Promoter mediated expression in a eukaryotic cell is mediated by three classes of RNA polymerases: RNA polymerase I (Pol I), characterized as transcribing most ribosomal RNAs (rRNAs); RNA polymerase II (Pol II), characterized as transcribing all polypeptide encoding sequences and most small nuclear RNAs (snRNAs); and RNA polymerase III (Pol III), characterized as transcribing 5S rRNA, transfer RNAs (tRNAs), U6 snRNA, and other small RNA gene products. Embodiments of the disclosure include a variety of Pol I and Pol II promoters as well as Pol III promoters like the 7SL RNA promoter (see Chu et al. Nucl. Acids Res. 1995, 23(10):1750-7; Bredow et al. Gene 1990 86(2):217-25; and Kleinert et al. J. Biol. Chem. 1988, 263(23):11511-5). Some embodiments of the disclosure include sequences, such as about 22 nucleotides of the human 7SL RNA coding sequence, as part of the 7SL RNA promoter.

A promoter is “endogenous” to a nucleic acid sequence (such as an operably linked coding region), and vice versa, where the combination is found in a naturally occurring nucleic acid molecule.

A promoter is “exogenous” or “heterologous” to a nucleic acid sequence, and vice versa, where the combination, in cis (on the same molecule) or in trans (on separate molecules), is not found in a naturally occurring nucleic acid molecule. Such a combination may also be referred to as a “recombinant” combination of nucleic acid sequences wherein human manipulation or intervention via recombinant DNA techniques and/or methods are used to combine the sequences. Non-limiting examples of recombinant techniques and methods include cleavage of nucleic acid molecules, ligation of nucleic acid molecules (such as of a promoter sequence to a coding sequence), insertion of a nucleic acid molecule into a vector, and transformation or transfection of a cell with an exogenous nucleic acid molecule.

The term “transfection” refers to the introduction of a nucleic acid molecule into a cell. Where the molecule is capable of expressing a gene product in the cell, the process is nucleic acid-mediated gene transfer. The term “transformation” refers to transfection wherein the recipient cell's genotype is changed as a result of the introduced molecule. Transformation may be mediated by a viral vector or particle which increases the efficiency of cellular uptake of an exogenous nucleic acid molecule. Either process may be used to deliver the nucleic acid molecules of the disclosure.

A “subject” refers to a human being or non-human animal or other “host” organism to be treated by a method of the disclosure. Non-limiting examples of a subject include non-human mammals, such as, but not limited to, primates, bovines, porcines, canines, felines, and rodents (e.g., mice and rats). Representative examples of an animal include vertebrates, sheep, elks, deer, mule deer, minks, mammals, monkeys, horses, cattle, pigs, goats, dogs, cats, rats, mice, birds, chickens, reptiles, and fish.

Sequence identity between two amino acid sequences may be expressed as a percentage with reference to the number of positions in the two optimally aligned sequences which have identical residues (times 100) divided by the number of positions compared. A gap, or a position in an alignment with a residue present in one sequence but not in the other, is treated as a position with non-identical residues. The alignment of the two sequences may be performed by a known algorithm, such as the Wilbur and Lipmann algorithm (Wilbur and Lipmann, 1983), using a window-size of 20 nucleotides or amino acids, a word length of 2 amino acids, and a gap penalty of 4. Computer-assisted analysis and interpretation of sequence data, including sequence alignment as described herein, can be conveniently performed using commercially available software packages such as the programs of the IntelligenetiCS™ Suite (Intelligenetics Inc., CA) or the GCG Wisconsin Package.

The phrase “hybridizes under stringent conditions” refers to the interaction between two single-stranded nucleic acids to form a double-stranded duplex molecule. The region of double-strandedness may be full-length for both single stranded molecules, full-length for one of the two single stranded molecules, or not full-length for either of the single-stranded nucleic acids. “Stringent conditions” refer to hybridization conditions comprising, or equivalent to, 68° C. in a solution consisting of 5×SSPE (0.9 M NaCl, 0.05 M NaH₂PO₄, 5.0 mM EDTA, pH 7.0), 1% SDS, 5×Denhardt's reagent and 100 μg/ml denatured salmon sperm DNA followed by washing in a solution comprising 0.1×SSPE, and 0.1% SDS at 68° C., or the above conditions with 50% formamide at 42° C. Stringent condition washes can include 0.1×SSC to 0.2×SSC, 1% SDS, 65° C., for about 15 20 min. A non-limiting example of stringent wash conditions is 0.2×SSC wash at 65° C. for about 15 minutes (see, Sambrook et al., Molecular Cloning—A Laboratory Manual (2nd ed.) Vol. 1-3, Cold Spring Harbor Laboratory, Cold Spring Harbor Press, NY, 1989, for a description of SSC buffer). Other exemplary stringent conditions include 7% SDS, 0.25 M sodium phosphate buffer, pH 7.0-7.2, 0.25 M sodium chloride at 65° C. to 68° C. or such conditions with 50% formamide at 42° C. Exemplary moderate or medium stringency conditions are as described above for stringent conditions but with 35% formamide at 42° C. is used, and the washes are carried out at about 55° C.

As used herein, “comprise” or “comprising” or “include” or “including” or variants thereof are used in the “open” sense such that the terms are inclusive and permit the presence of additional elements. The terms specify the presence of the stated features, steps, or components as recited without precluding the presence or addition of one or more features, steps, or components. Therefore a nucleic acid molecule comprising a) a first coding region encoding a RIG-I pathway agonist, and b) a second coding region encoding an antigen, may comprise more nucleotides than those actually cited. Thus the molecule may be part of a larger nucleic acid molecule.

General

The nucleic acids, compositions, and methods of the disclosure may be advantageously applied to enhance the potency of DNA vaccines by including a means for producing a RIG-I pathway agonist with the vaccine. Using a plasmid DNA (pDNA) vaccine as a non-limiting example, incorporation of one or more agonist coding regions in the same pDNA encoding the antigen of interest increases the potency of the pDNA vaccine.

The RIG-I pathway may be beneficially targeted given its broad cellular distribution (essentially every cell type) in different tissues, ability to increase IFN-β levels or production, and role in activating IRF3 and NFκB-dependent cytokine and chemokine expression. This is in sharp contrast to the narrow cellular distribution of TLRs (limited to professional antigen presenting cells or APCs, B lymphocytes, T lymphocytes, and natural killer or NK cells). Therefore, the methods of the present disclosure may be advantageously applied to the far more common TLR negative cells that may contain a DNA vaccine after vaccination, compared to TLR+ target cells.

The disclosed molecules, compositions, and methods may be used to enhance a subject's immune response, such as to an antigen of interest as described herein. The enhancement may be by use of a single vector molecule expressing both agonist and antigen, optionally via a single RNA transcript (that contains an RNA agonist and encodes an antigen or encodes both a polypeptide agonist and antigen), or more than one vector which express the agonist and antigen. The resulting stimulation or activation events of the adaptive immune responses (such as IFN-β production and/or the release of pro-inflammatory cytokines) by a cell are coordinated with the expressed antigen to potentiate an antigen-specific adaptive immune response in a subject.

Notably, the type I interferon (IFN)-dependent state produced, in a subject, with the molecules, compositions, and methods of the present disclosure may proceed to enhance T and B lymphocyte responses in the subject. In some embodiments of the disclosure, an IFN-dependent state may be produced via a disclosed method comprising a viral antigen to produce a protective response in a subject against the viral antigen or a virus that presents the antigen to the subject. Without being bound by theory, and offered to improve the understanding of the present disclosure, a kinetically-united expression of viral antigen and PAMP signaling will likely occur in the same cell, potentially simulating the milieu of a viral infection without the attendant immune evasion strategies and pathologic consequences of many viruses. The same benefit of reduced immune evasion and avoidance of pathology is believed to apply to other antigens, such as those of a microbial pathogen.

Nucleic Acids and Vectors

The disclosure includes nucleic acid molecules for the expression of RIG-I pathway agonists and antigens as described herein. The molecules may be considered a “vector”, which refers to a nucleic acid containing entity that may be used to transfect or transform a cell and into which a polynucleotide can be inserted. Non-limiting examples of a vector include a nucleic acid molecule per se, such as a plasmid (a circular, covalently closed double-stranded DNA loop) or linear expression construct (LEC), or a virus, such as a viral particle or virion. A linear construct is optionally a linearized circular construct, such as a plasmid that has been linearized by digestion with a restriction endonuclease.

A vector may be composed of DNA or RNA and is optionally a replicon, that may be maintained in a cell as an episome, via the presence of an origin of replication. In additional embodiments, a vector may be double-stranded or single-stranded. Additionally, a polynucleotide vector may comprise conventional phosphodiester bonds or include one or more non-conventional bond, such as an amide bond as found in peptide nucleic acids (PNA).

In most instances of the disclosure, a vector is an “expression vector” which permit transcription and translation of a nucleic acid inserted therein. In many embodiments, an expression vector comprises sequences that are heterologous to each other in that one or more of them are not found together in naturally occurring nucleic acids. An expression vector may be used to express a coding region encoding an agonist and/or antigen of the disclosure as well as any other gene product desired to be co-expressed with the agonist and/or antigen. Generally, a vector includes an assembly of genetically based regulatory element(s), such as promoters, operators, and/or enhancers, operatively linked to a coding region which is transcribed into RNA and/or translated into a polypeptide as described herein, and appropriate transcription and translation initiating and terminating elements (sequences). The choice of regulatory element(s) may vary according to the desired recipient cell as well as the desired level of expression.

One example of a vector of the disclosure include viral vectors such as those derived or based on a DNA virus, with adenovirus vectors, adeno-associated virus vectors, herpes virus vectors, vaccinia virus vectors, and poxvirus vectors as non-limiting examples. Other exemplary vectors include vectors derived from or based on an RNA virus, with alphavirus vectors, retrovirus vectors, and lentivirus vectors as non-limiting examples. Representative examples of positive sense RNA vectors include Sindbis virus vectors, VEE virus vectors, Semliki Forest virus vectors, Poliovirus vectors, and Kunjin virus vectors, while representative negative sense RNA vectors include Influenza virus vectors, Rabies virus vectors, Vesicular stomatitis virus vectors, Respiratory syncytial virus vectors, Sendai virus vectors, and SV5 vectors. While a viral vector, upon expression in a cell, may be able to give rise to infectious (attenuated) viruses, a vector of the disclosure may be a noninfectious replicon such that it is limited to replication (and expression of any foreign or heterologous sequences) in a transfected cell. In some cases, a viral vector may be packaged to allow it to infect a cell of the disclosure via a viral mechanism of infection.

In other embodiments, a RNA virus vector may comprise an RNA sequence which functions, without the need for translation, as a RIG-I pathway agonist of the disclosure. Non-limiting examples of vectors comprising an RNA sequence which functions as an agonist (with the need for translation of the sequence) include influenza based vectors, Sendai virus based vectors, flavivirus virus (such as yellow fever virus, dengue virus, Japanese encephalitis virus, and West Nile virus) based vectors. Of course an RNA virus vector embodiment of the disclosure may comprise a 5′ triphosphate.

In some embodiments, a nucleic acid molecule of the disclosure comprises, and is capable of expressing, a first coding region encoding a RIG-I pathway agonist and a second coding region encoding an antigen. The two coding regions are thus present on the same nucleic acid molecule. In DNA embodiments, the two coding regions maybe transcribed as a single RNA transcript (e.g. via a single transcription unit) or as two transcripts (e.g. via separate transcription units). The use of two transcription units on a single vector, such as a plasmid or linear construct, may be advantageously used based on its flexibility and adaptability. The first transcription unit, comprising the first coding region encoding a pathway agonist under the control of a first promoter, may be designed such that the first coding region may readily be substituted by inserting a sequence encoding another agonist. The first promoter may also be selected to be optimized for production of pathway agonists, such as RNA agonists of the pathway. Non-limiting examples include a first promoter that produces RNA transcripts with a 5′ triphosphate moiety, such as a Pol III promoter or the 7SL RNA promoter.

In a similar manner, the second transcription unit, comprising the second coding region encoding an antigen under the control of a second promoter, may be designed such that the second coding region may readily be substituted by inserting a sequence encoding another antigen. The second promoter may also be selected to be optimized for production of a polypeptide antigen. Non-limiting examples include a second promoter that is an Pol II promoter as described herein.

In additional embodiments, the RNA transcript may be translated to produce a polypeptide agonist and a polypeptide antigen. In other embodiments, the RNA transcript functions both as an RNA-based agonist and is translated in part to produce a polypeptide antigen.

DNA embodiments that express as a single RNA transcript comprise the first and second coding regions arranged in a tandem (head to tail) orientation. In some embodiments, the tandem orientation begins with the 5′ end of the coding region for the agonist followed by the 5′ end of the coding region for the antigen. In other embodiments, the tandem orientation begins with the 5′ end of the coding region for the antigen followed by the 5′ end of the coding region for the agonist. Where both coding regions are to be subsequently translated, the transcript may comprise an internal ribosome entry signal (IRES) to direct entry and translation of the coding region distal to the 5′ end of the transcript. An IRES may also be used where only the distal coding region is to be translated.

The disclosure also includes a first nucleic acid molecule comprising a coding region encoding a RIG-I pathway agonist, and a second nucleic acid molecule comprising a coding region encoding an antigen. Such an arrangement of coding regions may present the regions as separate transcription units on separate molecules. In other embodiments, one molecule may be an RNA molecule, such as an RNA virus vector, that is a RIG-I pathway agonist while the second molecule, such as a DNA or RNA virus vector, encodes the antigen. In some embodiments, the two molecules are used together to express the agonist and antigen as a pair of vectors. Given the separation of the coding regions on two vectors, each coding region may be operatively linked to its own promoter or regulatory element(s) in DNA vectors to control expression of the encoded agonist and antigen.

In some embodiments, the RIG-I pathway agonist encoded by a first nucleic acid molecule is an RNA agonist of the RIG-I pathway. In many embodiments, the RNA agonist is a RIG-I agonist. The coding region encoding the RNA agonist may be operatively linked to an RNA Pol III promoter, such as a 7SL RNA promoter which produces RNA transcripts comprising a 5′ triphosphate moiety. The RNA agonist encoding nucleic acid molecule may be used in a method as described herein. The method may comprise introducing the molecule into a cell of a subject under conditions wherein the nucleic acid is expressed in said cell. Optionally, the method further comprises introducing a second nucleic acid molecule comprising a coding region encoding, and capable of expressing, an antigen as described herein.

As noted above, the capability of a nucleic acid molecule of the disclosure to express a coding region is based upon the presence of a promoter, and any additional regulatory element(s), operatively linked to the coding region. A promoter is a nucleic acid sequence which regulates initiation, and possibly the rate, of transcription of a DNA sequence operably linked or positioned relative to the promoter. A promoter may contain sequences for the binding of regulatory proteins and other molecules, such as RNA polymerase and transcription factors as non-limiting examples. A skilled person would understand the term “operably linked” to refer to a correct location and/or orientation between a promoter and a nucleic acid sequence such that initiation of transcription by the promoter results in transcription of the nucleic acid sequence. So in embodiments where the nucleic acid sequence is a coding region, such as one containing an open reading frame, the promoter initiated transcription results in the production of an mRNA corresponding to the coding region. The mRNA may subsequently be translated to complete expression of the encoded protein.

The disclosure is not limited by the promoter(s) or other control sequences present in a described nucleic acid molecule. The range of promoters is described herein and include RNA polymerase I (Pol I), RNA polymerase II (Pol II), and RNA polymerase III (Pol III) promoters that are active in eukaryotic cells. Polypeptide-based agonists, and polypeptide-based antigens, of the disclosure may be operably linked to a Pol II promoter. RNA-based agonists of the disclosure are preferably operably linked to a Pol III or a Pol I promoter.

In some embodiments, a promoter of the disclosure is a recombinant or heterologous promoter relative to the coding sequence operatively linked thereto. A skilled person would understand a recombinant promoter to include a synthetic promoter that does not occur in nature and a heterologous promoter to include one that is not normally associated with the operatively linked coding sequence in its natural environment. Alternatively, a promoter is that which is normally found with a coding sequence in its natural environment, such as a promoter present in the 5′ non-coding sequences located upstream of a coding sequence. While such a promoter may be considered “endogenous” relative to the coding sequence, it may be isolated with the linked coding sequence or separately isolated and then recombinantly linked to the coding sequence.

Of course, the promoter of the disclosure will be that which is suitable for expressing the operatively linked coding region in a cell of interest, such as that of an animal or human subject as described herein. The skilled person has knowledge of, and may readily select, promoter/cell type combinations which allow for protein expression in a given cell. Non-limiting examples of a RNA Pol II promoter of the disclosure may be constitutive, tissue-specific, inducible, and/or otherwise able to direct high level expression of an operatively linked coding sequence. In some embodiments of the disclosure, the promoter is a human or simian cytomegalovirus (CMV) promoter capable of initiating expression in a variety of eukaryotic cells, including human cells. Other non-limiting examples of promoters include a Rous sarcoma virus (RSV) promoter, an actin promoter, a keratin promoter, a ubiquitin promoter, or an SV40 promoter.

In further embodiments, a promoter is one which allows tissue specific expression, such as a hypoxia-induced promoter, a tumor-specific promoter, a skeletal actin promoter, or a myosin promoter. In alternative embodiments, the promoter is supercoiling independent, stronger than a CMV promoter on a linear template (such as in a eukaryotic cell), or weaker than a CMV promoter on a linear template (such as in a eukaryotic cell).

As described herein, two promoters may be used to regulate the first and second coding regions of the disclosure. If present on a single nucleic acid molecule, the resultant vector would be bi-cistronic. Non-limiting examples include the use of two or more promoters selected from the human or simian CMV, RSV, or SV40 promoters in a vector to express the disclosed agonist and antigen. In some embodiments, the first and second coding regions are operably linked to the human and simian CMV promoters, or to the simian and human CMV promoters, respectively. Such an arrangement may allow for the production of a ratio of transcripts of the first and second coding regions within a cell. Other ratios of the two coding regions or their products are described below.

In embodiments comprising expression of an RNA agonist with a 5′ triphosphate moiety, the coding region may be operably linked to a Pol III promoter, such as a 7SL RNA promoter.

In additional embodiments of the disclosure, a vector comprises one or more regulatory sequences or elements for use in conjunction with a promoter. In some cases, the regulatory sequence is an enhancer, which a skilled person would recognize as a cis-acting element involved in transcriptional activation. An enhancer may be a recombinant or heterologous enhancer, such as one not normally associated with a disclosed promoter or coding sequence in its natural environment. Such an enhancer may be a synthetic sequence or a sequence associated with another coding sequence. Enhancers for use in a disclosed vector may be isolated from any suitable source. Alternatively, an enhancer may be one normally located either downstream or upstream of a disclosed promoter or coding sequence in its natural environment.

In alternative embodiments, an additional regulatory sequence may be a reversible repressor or activator that may be used to regulate expression from a vector. Non-limiting examples include a Tet responsive element, an ecdysone response element, an antiprogestin-inducible element, an oxygen level responsive element, or an antibiotic responsive element. In some cases, a hypoxia-inducible promoter may be used in a vector suitable for transforming cells, such as some tumor cells, that are under hypoxic conditions.

A linear vector of the disclosure may be used for expression as described herein without the presence of a termination signal to end transcription. In such embodiments, transcription may be considered “run-off” such that an RNA polymerase continues transcription until it reaches the end of the vector. In other embodiments, a vector comprises one or more termination signal and/or a polyadenylation signal.

A termination signal or terminator is a DNA sequence directing termination of an RNA transcript by an RNA polymerase. The presence of a termination signal ends production of an RNA transcript. In eukaryotic cells, a termination signal or region of sequence may also contain sequences that expose a polyadenylation site on the transcript to allow the addition of a polyadenylate (polyA) tail. This results in the production of polyadenylated mRNA, which may be more stable and more efficiently transferred from a cell's nucleus to the cytoplasm or more efficiently translated in a eukaryotic cell. Therefore, and in some embodiments involving eukaryotic cells, such as human cells, a vector of the disclosure includes a polyadenylation signal. Non-limiting examples of a termination signal and/or a polyadenylation signal include that of bovine growth hormone (BGH), rabbit beta globin, or simian virus 40 (SV40).

An additional non-limiting examples of sequences that may be present in cis on a vector include a non-coding sequence between the promoter and the coding sequence, such as a 5′ untranslated region, and an intron sequence within a coding sequence. In the cases of the latter, the vector is one which is for use in a cell, such as a eukaryotic cell, which correctly removes the intron prior translation. Moreover, appropriate donor and/or acceptor splicing sites are introduced as needed to ensure proper excision of introns and post-transcriptional processing for expression of an encoded polypeptide.

A disclosed vector may further comprise an initiation of translation signal and/or an internal ribosome binding site (IRES). In some embodiments, an LEC includes an ATG initiation codon that is “in-frame” with the ORF of a coding sequence as described herein to facilitate translation of an encoded polypeptide. In other embodiments, an IRES is present in a vector to allow expression of more than one ORF from a single transcript expressed from a vector's coding sequence. Thus, a polycistronic vector is within the scope of the disclosure. IRES elements able to begin translation at an internal site of an mRNA molecule have been reported. Non-limiting examples of IRES elements include those from members of the picornavirus family, namely polio and encephalomyocarditis, and a mammalian IRES element. Because an IRES element can be linked to a heterologous ORF, more than one ORF can be expressed as a single transcript, but each separated by an IRES element, and then used as a polycistronic message to express the polypeptides encoded by the ORFs. Thus more than one ORF can be efficiently expressed using a single promoter. See, for example, U.S. Pat. Nos. 5,925,565 and 5,935,819.

In further embodiments, a vector may comprise one or more additional control sequences, such as those beyond the elements mentioned above, and which participate in regulating or directing transcription and possibly translation of an operably linked coding sequence in a particular host organism. A non-limiting example includes the first and second translation enhancing elements as described in WO 97/49814. While a promoter and a terminator may be considered minimal control sequences, other control sequences that govern transcription and/or translation may be used.

A representative, and non-limiting example of an expression cassette, optionally for use as a vector of the disclosure, encoding a polypeptide RIG-I agonist is presented as follows. In one embodiment, a coding sequence containing the CARD portion of full-length RIG-I is placed into a standard 1055-1 pDNA backbone (containing the human CMV IE-1 promoter/enhancer and intron A, followed by a multiple cloning site for insertion of a gene of interest, followed by a rabbit beta globin termination/polyadenylation sequence) with the influenza M2 gene as the gene of interest (VR4759). The pDNA is modified to insert the PS3 IRES sequence followed by a ΔRIG-I coding sequence (such as a sequence encoding residues 1-264 of SEQ ID NO:1 herein). Alternatively, the modification is to insert the IRES sequence followed by a ΔRIG-Istop coding sequence, which contains 1 or more stop codons to render the expressed polypeptide inactive and to provide a negative control pDNA. The stop codon(s) are optionally added via a 5′ PCR amplification oligonucleotide primer used to prepare the coding sequence. A further discussion of these constructs is provided in Examples 4 and 5 herein.

Overexpression of ΔRIG-I has been reported to activate the IFN pathway (Yoneyama, Nat Imm 2004). CARD activation results in phosphorylation of IKKε and tank binding kinase 1 (TBK 1) which subsequently leads to activation of transcription factors IRF3 and NFκB, and then to the production of type I IFN as well as other pro-inflammatory cytokines (Kato Immunity 2005).

In additional embodiments, the polypeptide agonist is MAVS, such as SEQ ID NO:2 herein. Thus an MAVS coding region is inserted into a construct as described above and in place of the ΔRIG-I or ΔRIG-Istop coding sequence.

A representative, and non-limiting example of a dsRNA RIG-I agonist-expressing cassette is presented as follows. The 5′ and 3′ untranslated regions (UTRs) of HCV contain dsRNA motifs that have been reported to induce RIG-I dependent IFN production (Sumpter, J. Virology 2005). These dsRNA species may be expressed by either the same or a separate nucleic acid molecule than a RIG-I pathway agonist. One straightforward and versatile design for RIG-I agonist co-expression will be to dedicate a first transcription unit, and so first transcript, of a nucleic acid molecule to the agonist and a second transcription unit, and so second transcript, to the antigen. Using a 1055-1 backbone, a loci distinct from the RNA polymerase II expression cassette can be used for introducing an RNA polymerase III expression cassette. Both orientations of the RNA Pol III cassette relative to the RNA Pol II cassette may be prepared. The RNA Pol III cassette is preferred for expression of a dsRNA agonist because it (1) produces RNA transcripts which lack a 7-methyl guanosine CAP and a poly A+ tail; (2) utilizes a simple but strong transcription termination signal (series or sequence of TTTT's); (3) has a high transcription rate, efficient nuclear export, and high RNA stability potential; (4) presents a small footprint on the pDNA backbone and potential for encoding multiple copies; and (5) has a propensity for creating RNA with double stranded structure(s). Pol III promoters that produce RNAs with a 5′ triphosphate moiety may be advantageously used for embodiments of the disclosure comprising the moiety. In some cases, the Pol III promoter is the 7SL RNA promoter, optionally comprising a necessary portion of the transcribed 5′ 7SL RNA sequence.

The promoters for RNA Pol III, which transcribes tRNA, are small elements of about 120-140 nucleotides containing an A and B box with specific sequences which lie internal to the +1 transcript start site. This system advantageously offers great flexibility in creating various dsRNA-containing transcripts that can be used both for assay in vitro to identify highly stimulatory RNA species.

Compositions

The disclosure also includes a composition or formulation containing one or more nucleic acid molecule as described herein. In some embodiments, the composition comprises a single molecule comprising the first and second coding regions as described herein. In other embodiments, the composition comprises a pair of nucleic acid molecules that contain the two coding regions. In cases comprising a pair of molecules, the molar quantity of each molecule may be identical or different. A difference in the molar quantity of the molecules may be expressed as a molar ratio of the first coding region to the second coding region. The ratio may range from about 10:1, about 9:1, about 8:1, about 7:1, about 6:1, about 5:1, about 4:1, about 3:1, about 2:1, about 1:2, about 1:3, about 1:4, about 1:5, about 1:6, about 1:7, about 1:8, about 1:9, or about 1:10 of the first to second coding regions. In cases wherein a first molecule comprises more than one agonist encoding sequence, or a second molecule comprises more than one antigen encoding sequence, the molar ratio may still be used by a skilled person to select the relative amounts of the nucleic acid molecules to use in the practice of the disclosure. A similar ratio may be determined and selected for use based upon the relative strengths of the promoters used to transcribe the two coding regions and/or the relative efficiency of expressing (through transcript stability, nuclear export, and/or translation, folding, and post-translational modification) the encoded agonist and antigen.

A composition of the disclosure includes a particle, bead, polymer or suspendable solid support removably associated with one or more nucleic acid molecules. Non-limiting examples include a particle, bead, or polymer which is removably associated with a nucleic acid molecule of the disclosure so as to facilitate its handling or transfer properties. The association with a particle, bead, polymer or suspendable solid support may be via covalent and/or non-covalent means. In cases of association mediated by a covalent bond, the bond may be one which is cleaved by an enzymatic activity, such as an enzyme that is present in a target cell, or an animal or human subject, to which the composition is delivered.

In cases of association mediated by non-covalent interactions, the association may be that which is sufficiently stable to remain until after delivery of the composition to a target cell, such as that in an animal or human subject. Thus, in some embodiments, the composition is sufficiently stable to resist separation of the nucleic acid molecule and the solid support until after introduction into the target cell. In other embodiments, the composition allows separation of the molecule and the solid support after delivery to an animal or human subject such that the molecule may be taken up by a target cell.

In some embodiments, a particle (such as a microparticle or nanoparticle) or bead may be made of, or coated with, gold and/or tungsten prior to association with one or more nucleic acid molecules. The association includes specific or non-specific conjugation of the molecule to a suspendable solid support. Additional embodiments include a solid lipid nanoparticle (SLN) or cationic SLN (see for example Pedersen et al. Eur. J. Pharm. Biopharm. 2006, 62(2):155-62); gelatin nanoparticles (see for example Zwiorek et al. J. Pharm. Pharm. Sci. 2005, 7(4):22-8); nanoparticles (see for example Prow et al. Mol. Vis. 2006, 12:606-15); and magnetic particles or beads (see for example Day et al. Biochem. J. 1991, 278(Pt. B):735-40). Further embodiments include a solid support suitable or compatible for use with a bolistic delivery system, such as a commercially available “gene gun,” or other microprojectile bombardment means.

The number of copies of nucleic acid molecules on each solid support may vary as permitted by the chemistries of the solid support and the means of association. In some embodiments, such as with a pair of molecules containing the first and second coding regions as described herein, more than one molecule is associated with each solid support. So embodiments of the disclosure include about 2, about 4, about 6, about 8, about 10, about 12, about 14, about 16, about 18, about 20 or more copies of a nucleic acid molecule with each particle, bead, polymer or other solid support medium.

In further embodiments, a linear nucleic acid molecule may be attached to a suspendable solid support via the 5′ end of a primer used to synthesize the molecule. In some cases, the primer is attached to the solid support prior to its use in a polymerization reaction to produce the nucleic acid molecule. The polymerization reaction may be polymerase-mediated or by de novo synthesis. In alternative embodiments, a suspendable solid support is attached to a 3′ end of a linear nucleic acid molecule. Non-limiting examples include use of a disulfide thiol modification to introduce a 3′-thiol linkage, a 3′-amine modifier to introduce a primary amine moiety, and the addition of a 3′-phosphate group to the 3′ end.

In another aspect, the disclosure includes a composition comprising a carrier associated with one or more nucleic acid molecules as disclosed herein. Non-limiting examples of a carrier include mono- or poly-cationic molecules which can complex with the molecule via ionic interactions. In some embodiments, the cationic molecule is a cationic lipid such that the carrier is a lipid vesicle or lipoplex comprising cationic lipids. In other embodiments, the cationic molecule is a polymer or polypeptide such that the carrier is a polyplex carrier comprising cationic polymers (see for example, Kodama et al. Curr Med Chem. 2006, 13(18):2155-61).

A lipid vesicle carrier of the composition may contain lipids suitable for the formation of vesicles, such as, but not limited to, a cationic lipid and/or a neutral lipid. Non-limiting examples of vesicles include unilaminar vesicles, including micelles and liposomes, and multilamellar vesicles (MLVs), or combinations thereof. In some embodiments, the vesicle contains both a cationic and a neutral lipid, optionally in equimolar amounts. In some cases, the cationic lipid is VC1052 and the neutral lipid is DPyPE.

Embodiments of the disclosure include a lipid vesicle carrier containing one or more nucleic acid molecules within the lumen of the vesicle as well as a lipid vesicle carrier that has the molecule associated with the lipid layer or exterior of the carrier. Of course combinations of such carriers are also within the scope of the disclosure. As a non-limiting, representative example, a nucleic acid molecule of the disclosure may be contained within a liposome, which is composed of a phospholipid bilayer membrane enclosing an inner space or lumen, which is commonly aqueous in character. Alternatively, the molecule may be present in the bilayer, or on the exterior, of the liposome. Of course, the molecules may be present in more than one of these possible liposomal locations.

A multilamellar vesicle, or liposome, has multiple lipid layers separated by medium contained within intra-layer space, which is also commonly aqueous in character. In some embodiments, a nucleic acid molecule is contained within the intra-layer space of a multilamellar vesicle. In some cases, a molecule is complexed with Lipofectamine (Invitrogen). Alternatively, the molecule is complexed with a cationic polymeric dendrimer, such as Superfect (Qiagen).

The molar ratio of nucleic acid molecule(s) to cationic lipid or total lipid in a composition may range from about 8:1 to about 1:8, or even higher proportion of lipid. In some embodiments, the molar ratio is about 7:1, about 6:1, about 5:1, about 4:1, about 3:1, about 2:1, about 1:1, about 1:2, about 1:3, about 1:4, about 1:5, about 1:6, or about 1:7.

In further embodiments of the disclosure, a vesicle may be complexed with a hemagglutinating virus of Japan (HVJ), which has been shown to facilitate fusion with a cell membrane. This would promote cell entry of a liposome-encapsulated nucleic acid molecule.

A nucleic acid containing composition of the disclosure is optionally lyophilized or vitrified, such as for purposes of storage or increasing stability. Lyophilization (freeze-drying) and vitrification, to form a glass-like frozen solid without the formation of ice crystals, may be by any means known to the skilled person.

Additionally, the disclosure includes formulations of the described nucleic acid molecules in a unit dosage form for inducing, promoting, or producing an immune response, such as a protective response, against an antigen as described herein. In some embodiments, the formulation comprises a pharmaceutically acceptable carrier or excipient, optionally with an adjuvant. A unit dosage is an amount which is sufficient and/or effective to induce, promote, or produce an immune response in a treated animal or human subject. The dosage may vary depending on a variety of factors, including, as non-limiting examples, the promoter used, the characteristics of the coding sequence, the method of delivery, and the weight and type of the treated subject. Nonetheless, the unit dosage form for disclosed nucleic acid molecule and a subject may be readily determined by limited routine and repetitive study as known to the skilled person.

Non-limiting examples of amounts include about 1 nanogram to about 5 milligrams, although about 10 ng, about 20 ng, about 30 ng, about 40 ng, about 50 ng, about 75 ng, about 100 ng, about 125 ng, about 150 ng, about 200 ng, about 250 ng, about 300 ng, about 350 ng, about 400 ng, about 450 ng, about 500 ng, about 550 ng, about 600 ng, about 650 ng, about 700 ng, about 750 ng, about 800 ng, about 850 ng, about 900 ng, about 900 ng, about 950 ng, about 1 μg, about 5 μg, about 10 μg, about 20 μg, about 30 μg, about 40 μg, about 50 μg, about 75 μg, about 100 μg, about 125 μg, about 150 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 900 μg, about 950 μg, about 1 mg, about 2 mg, about 3 mg, about 4 mg, or about 5 mg or more of a nucleic acid molecule may be delivered in the practice of the disclosure. The above described amounts may be of isolated plasmid or linear nucleic acid molecules or of nucleic acid molecule(s) in a packaged viral vector.

As noted above, a disclosed composition may further comprise a pharmaceutically acceptable excipient or carrier for delivery of the nucleic acid molecule to a cell or a subject. Such an acceptable excipient or carrier includes a pharmaceutically-acceptable material, composition or vehicle. Representative embodiments include a liquid or solid filler, diluent, solvent or encapsulating material. Non-limiting examples of acceptable excipients and carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances acceptable for use in pharmaceutical formulations.

In some embodiments, the excipient or carrier solubilizes a nucleic acid molecule of the disclosure. Non-limiting examples of such an excipient or carrier include various buffers, optionally mixed or complexed with adjuvant components, such as cytofectins and co-lipids. Some examples of buffers are phosphate buffered saline (PBS), normal saline, Tris buffer, and sodium phosphate, with the pH adjusted as desired or appropriate. In cases of an insoluble polynucleotide, additional of a weak acid or weak base followed by dilution to a desired volume with a buffer may be used. In additional embodiments, a pharmaceutically acceptable additive can be used to provide a desired osmolarity.

The disclosure includes complexing of a described nucleic acid molecule with one or more adjuvant components by any means known in the art. Representative examples include mixing a pDNA solution and a solution of cytofectin/co-lipid liposomes. In one embodiment, the concentration of each of the constituent solutions is adjusted prior to mixing such that the desired final pDNA/cytofectin:co-lipid ratio and the desired pDNA final concentration will be obtained upon mixing the two solutions. For example, if the desired final solution is to be physiological saline (0.9% weight/volume), both pDNA and cytofectin:co-lipid liposomes are prepared in 0.9% saline and then simply mixed to produce the desired complex. Alternatively, a cytofectin:co-lipid liposome can be prepared by hydrating a thin film as known in the art.

The molecules, vectors, compositions, and formulations of the disclosure may be considered therapeutic agents in that they may be delivered to a cell or subject as described herein to result in a therapeutic effect. Thus the disclosure includes DNA and RNA molecules as therapeutic agents.

Agonists

The disclosure includes RIG-I pathway agonists that are RNA or polypeptide molecules. In some embodiments, the agonist is an agonist of RIG-I activity in the pathway, such as an RNA molecule that interacts with RIG-I protein to activate the pathway in whole, to result in IFN-β production, or in part, such as to activate the NFκB and/or interferon regulatory factor 3 (IRF3) signaling pathways. Of course the disclosure includes the possibility of using two or more agonists together, such as by use of more than one first coding region to encode more than one agonist.

In some embodiments, an RNA agonist of RIG-I protein agonist is a dsRNA or ssRNA. In some cases, the RNA agonist comprises a 5′ triphosphate moiety. The RNA agonist may be encoded by a coding region as described herein, such as under the control of a RNA Pol III promoter to produce a 5′ triphosphate moiety as a non-limiting example. In some cases, a dsRNA contains a motif found as all or part of a dsRNA replication intermediate in an RNA virus. Such a motif may be readily identified as a potent IFN inducer. One available assay is based upon in vitro screening to establish that type I IFN pathways are activated following intracellular expression or introduction of a dsRNA molecule. Candidate cell lines (which may be previously screened to be TLR3 negative) include human fibroblasts, HEK293 cells, and, if a muscle cell representative is desired, human RD cells. IFN-β is one cytokine candidate that can be detected in cultures of fibroblasts activated by a RIG-I agonist (Yoneyama, 2004; Seth, Cell 2005).

Embodiments of a dsRNA agonist include those that contain a selected sequence as well as a particular RNA length or limited range of lengths. Without being bound by theory, and offered to improve to the understanding of a dsRNA agonist, different selected sequences may have different affinities for binding to the RNA helicase domain of RIG-I protein which result in variable efficiencies for inducing IFN production. So the readily available assays to determine or measure an agonist's ability to induce IFN production in vitro may be a means to infer the different binding affinities of an agonist to the RNA helicase or other dsRNA binding domain of RIG-I protein.

The disclosure includes the use of dsRNA agonists that are of a length insufficient to activate protein kinase R (PKR) pathways. Long dsRNA stretches have been reported to preferentially activate PKR pathways and lead to destruction of the dsRNA molecule as well as activate a pro-apoptotic pathway in the cells expressing the molecule. Embodiments of the disclosure include sufficiently short dsRNA agonists that are not subject to such degradation and subsequent activity, which may prematurely curtail IFN-β production, an enhanced immune response, and/or antigen expression as described herein. On the other hand, pro-apoptotic signals may enhance immune responses. Therefore, other embodiments of the disclosure include dsRNA agonists that are of a sufficient length to activate a pro-apoptotic signal or pathway without significant detriment to IFN-β production or enhancement of an immune response. Moreover, and due to the presence of Dicer and other components of the small interfering (si) RNA pathway in cells, the sequence of a candidate dsRNA agonist may be screened to avoid or remove sequences that potentially result in “knockdown” of genes within a cell to be treated with the agonist.

In additional embodiments, an RNA agonist of RIG-I protein agonist is a ssRNA, optionally with a 5′ triphosphate moiety. As in cases of a dsRNA, an ssRNA agonist may contain a motif found as all or part of a ssRNA or dsRNA replication intermediate in an RNA virus. Like with a dsRNA agonist, such an ssRNA motif may be readily identified as a potent IFN inducer. Also like with a dsRNA, an ssRNA agonist may contain a selected sequence as well as a particular RNA length or limited range of lengths.

Non-limiting examples of an RNA agonist include the sequence of the 5′ UTR and 3′ UTR of HCV as well as agonist fragments of these sequences. The 5′ UTR sequence is represented by SEQ ID NO: 3 herein, which contains positions 1-341 of the HCV genome (from Jopling, Science, 2005). While all of SEQ ID NO:3 may be transcribed into an RNA molecule for use as an agonist, embodiments of the disclosure include transcription of the first 130 bases of SEQ ID NO:3 into RNA for use as an agoinst. Coding regions for these sequences may be inserted into a nucleic acid molecule of the disclosure and expressed in the described methods to result in the production of an RNA RIG-I agonist. Fragments of these sequences, such as those truncated from one or both ends may also be prepared and expressed to produce additional RNA agonists. In other embodiments, these sequences comprise limited substitutions which do not affect the agonist activity of the transcribed RNA. Non-limiting examples include sequence substitutions to optimize expression of a sequence, such as mutation to remove a transcription termination sequences. Of course substitutions may be of any base that is non-essential for agonist activity, optionally while maintaining the RNA's secondary structure.

In embodiments with an HCV 5′ UTR, the 5′ UTR coding sequence is truncated at the 3′ end by about 1, about 3, about 5, about 7, about 9, or about 11 nucleotides such that the expressed RNA agonist sequence is shortened by the number of removed nucleotides. Of course the 5′ UTR coding sequence may be truncated at the 5′ end by about 1, about 2, about 3, or about 4 nucleotides. In other embodiments, the 5′ and/or 3′ end of SEQ ID NO:3 is shortened by about 1, about 3, about 5, about 7, about 9, about 11, about 13, about 15, about 17, about 19, about 21, about 23, about 25, about 27, about 29, about 31, about 33, about 35, about 37, about 39, about 41, about 43, about 45, about 47, about 49, about 51, about 53, about 55, about 57, about 59, about 61, about 63, about 65, about 67, about 69, or about 71 nucleotides.

In additional embodiments, the nucleotide sequence encoding an HCV 5′ UTR based agonist of the disclosure has about 99, about 98, about 97, about 96, about 95, about 94, about 92, about 90, about 88, about 86, about 84, about 82, or about 80 percent identity to SEQ ID NO:3 or a fragment thereof as described above. Such homologous sequences may be encoded by nucleic acid coding sequences that hybridize to SEQ ID NO:2 or a fragment thereof under stringent conditions. Alternatively, the agonist may be encoding by a sequence with about 85, about 80, about 75, about 70, about 65, or about 60 percent identity to all or part of SEQ ID NO:3. Such homologous nucleic acid sequences may hybridize to SEQ ID NO:3 or a fragment thereof under moderate or medium stringency conditions as described herein.

In further embodiments of an RNA agonist, an oligonucleotide sequence of about 20, about 25, about 30, about 35, about 40, about 45, or about 50 bases may be used. Non-limiting examples include RNAs comprising AGCUUAACCUGUCCUUCAA (SEQ ID NO:4), GGGGCUGACCCUGAAGUUCAUCUU (SEQ ID NO:5), GGGGAUGAACUUCAGGGUCAGCUU (SEQ ID NO:6), or GGGAGACAGGCACCACACACACACACACUUU (SEQ ID NO:7) at the 5′ end with a 5′ triphosphate moiety. Such an RNA agonist may be encoded by a first coding region as described herein. In alternative embodiments, the RNA agonist sequence may be placed at the 5′ end of an ssRNA vector, or either (or both) 5′ ends of a dsRNA vector, as described herein, to act as an RNA agonist per se. In such a case, the sequence of the remainder of the vector is optionally examined to remove or modify sequences predicted to form undesirable secondary structures with the above sequences. Of course the RNA vector may also optionally include a coding region encoding an antigen as described herein.

In some cases, an RNA vector comprises one 5′ end (or optionally two in the case of a dsRNA vector) with a 5′ triphosphate moiety and a double stranded sequence formed by pairing SEQ ID NOs: 5 and 6 via basepair complementarity. The resultant duplex sequence may be used in either orientation at a 5′ end of an RNA vector. And while the duplex molecule formed by the annealing of SEQ ID NOs: 5 and 6 has a single base 5′ overhang, embodiments of the disclosure include the optional “filled in” of the overhang by extending the 3′ end of SEQ ID NO:5 or 6 (or both) with an additional “U”.

Alternatively, an oligonucleotide RNA agonist as described above may comprise any one of SEQ ID NOs:4-7 truncated at the 3′ end by about 1, about 3, about 5, about 7, or about 9 nucleotides. Such a truncated RNA agonist may be encoded by a coding region, or incorporated as part of an RNA vector, as explained herein. In additional embodiments, an oligonucleotide RNA agonist has about 95, about 94, about 92, about 90, about 88, about 86, about 84, about 82, about 80, about 75, or about 70 percent identity to any one of SEQ ID NOs:4-7 or a truncated form thereof. Such homologous sequences may also be identified as, or encoded by, nucleic acid coding sequences that hybridize to any one of SEQ ID NOs:4-7 or a truncated form thereof under stringent conditions.

In alternative embodiments, an agonist of RIG-I protein is a polypeptide that interacts with RIG-I and/or MAVS to activate the RIG-I pathway in whole or in part. The agonist may modulate intracellular interactions between the RIG-I and MAVS proteins, or directly interact with MAVS (optionally as a competitor to RIG-I protein interactions therewith), to result in IFN-β production or activate the NFκB and/or interferon regulatory factor 3 (IRF3) signaling pathways. In some embodiments, a polypeptide agonist is a fragment of the RIG-I protein which activates the pathway, optionally via interaction with MAVS protein. One non-limiting example of such a fragment is a truncated form of RIG-I protein (ΔRIG-I) containing the 2 CARDs within the N-terminal 264 amino acids (residues 1-264 of SEQ ID NO:1). Any nucleotide sequence encoding such a polypeptide agonist, and suitable for expression in a desired cell or subject, may be used as an agonist coding region of the disclosure.

Given the range of variation in the RIG-I protein sequence between human subjects and other subjects of the disclosure, the disclosure includes the treatment of cells and subjects with RIG-I proteins that are encoded in nature by a nucleic acid sequence with one or more nucleotide polymorphisms relative to a sequence encoding SEQ ID NO:1 or have homology, but not identity, with SEQ ID NO:1 and with RIG-I fragments with high degrees of homology to fragments of SEQ ID NO:1. Thus the RIG-I proteins in cells and subjects of the disclosure may have sequences that have about 99, about 98, about 97, about 96, about 95, about 94, about 92, about 90, about 88, about 86, about 84, about 82, or about 80 percent identity to SEQ ID NO:1. Similarly, a RIG-I protein fragment agonist of the disclosure may have about 99, about 98, about 97, about 96, about 95, about 94, about 92, about 90, about 88, about 86, about 84, about 82, or about 80 percent identity to positions 1-264 of SEQ ID NO:1. Such homologous polypeptides may be encoded by nucleic acid coding sequences that hybridize to SEQ ID NO:1 or a fragment thereof under stringent conditions.

In additional embodiments, a RIG-I protein fragment agonist of the disclosure may have about 85, about 80, about 75, about 70, about 65, or about 60 percent identity to positions 1-264 of SEQ ID NO:1. Such homologous polypeptides may be encoded by nucleic acid coding sequences that hybridize to SEQ ID NO:1 or a fragment thereof under moderate or medium stringency conditions as described herein.

In other embodiments, a RIG-I pathway agonist is an agonist of MAVS protein activity which leads to IFN-β production. One non-limiting example of an MAVS agonist is the MAVS protein per se when overexpressed in a cell. Overexpression refers to an increase in the cellular content of MAVS such that the RIG-I pathway is activated. In other examples, an MAVS agonist is a protein fragment of the MAVS protein, such as SEQ ID NO:2, which activates the pathway and/or directly or indirectly activates the NFκB and/or interferon regulatory factor 3 (IRF3) signaling pathways. Any nucleotide sequence encoding such a protein agonist, and suitable for expression in a desired cell or subject, may be used as an agonist coding region of the disclosure. An MAVS protein fragment may be encoded by a nucleotide sequence that is truncated from one or both ends such that it encodes a polypeptide that is shortened by about 2, about 4, about 6, about 8, about 10, about 12, about 14, about 16, about 18, about 20, about 22, about 24, about 26, about 28, about 30, about 32, about 34, about 36, about 38, about 40, about 42, about 44, about 46, about 48, about 50, about 52, about 54, about 56, about 58, about 60, about 62, about 64, about 66, about 68, about 70, about 72, about 74, about 76, about 78, about 80, about 82, about 84, about 86, about 88, about 90, about 92, about 94, about 96, about 98, or about 100 amino acids.

In additional embodiments, a nucleotide sequence encoding an agonist of MAVS encodes a polypeptide that has about 99, about 98, about 97, about 96, about 95, about 94, about 92, about 90, about 88, about 86, about 84, about 82, or about 80 percent identity to SEQ ID NO:2 or a fragment thereof as described above. Such homologous polypeptides may be encoded by nucleic acid coding sequences that hybridize to SEQ ID NO:2 or a fragment thereof under stringent conditions. Alternatively, a MAVS agonist of the disclosure may have about 85, about 80, about 75, about 70, about 65, or about 60 percent identity to all or part of SEQ ID NO:2. Such homologous polypeptides may be encoded by nucleic acid coding sequences that hybridize to SEQ ID NO:2 or a fragment thereof under moderate or medium stringency conditions as described herein.

Antigens

An antigen as described herein may be any of interest and to which an immune response in a subject is desirable. Of course the disclosure includes the possibility of using two or more antigens together, such as by use of more than one second coding region to encode more than one antigen. An antigen may be an “immunogen”, which is any immunogenic polypeptide with one or more epitopes or combinations of epitopes that generates an immune response. An immunogen stimulates the immune system of a subject such that one or more functions of the immune system are increased (compared to the absence of the immunogen in the subject) and directed towards the immunogenic agent. An immunogen, whether alone or linked to a carrier in the presence or absence of an adjuvant, thus elicits a cellular and/or humoral immune response.

Both antigenic and immunogenic polypeptides react with the immune system of a vertebrate when introduced thereto. But while an immunogenic polypeptide will likely be antigenic, an antigenic polypeptide may not be immunogenic due to its size and/or conformation. Non-limiting examples of antigenic and immunogenic polypeptides include, but are not limited to, polypeptides or fragments thereof from infectious agents such as viruses, bacteria, fungi or other pathogens or parasites; allergens such as that from plants, pollen, grass, trees, ragweed, pet dander, dust, and other environmental sources; and some self antigens, such as tumor or cancer associated antigens.

Non-limiting examples of viral antigenic and immunogenic polypeptides include, but are not limited to, adenovirus polypeptides, alphavirus polypeptides, calicivirus polypeptides, e.g., a calicivirus capsid antigen, coronavirus polypeptides, distemper virus polypeptides, Ebola virus polypeptides, enterovirus polypeptides, flavivirus polypeptides, hepatitis virus (AE) polypeptides, a hepatitis B virus core or surface antigen, a hepatitis C virus antigen, herpesvirus polypeptides, a herpes simplex virus or varicella zoster virus glycoprotein, immunodeficiency virus polypeptides, the human immunodeficiency virus (HIV) envelope or protease, infectious peritonitis virus polypeptides, influenza virus and other orthomyxovirus polypeptides, an influenza (optionally type A) hemagglutinin (HA), neuramimidase (NA), nucleocapsid (NP), M2 polypeptide, or nucleoprotein, a lentivirus polypeptide, leukemia virus polypeptides, Marburg virus polypeptides, papilloma virus polypeptides, polypeptides, a hemagglutinin/neuramimidase polypeptide, parainfluenza virus, respiratory syncytial virus, measles virus polypeptides, and other paramyxovirus polypeptides, parvovirus polypeptides, pestivirus polypeptides, picoma virus polypeptides, a poliovirus capsid polypeptide, pox virus polypeptides, a vaccinia virus polypeptide, rabies virus polypeptides, a rabies virus glycoprotein G, reovirus polypeptides, retrovirus polypeptides, rotavirus polypeptides, and rotavirus polypeptides.

Non-limiting examples of bacterial antigenic and immunogenic polypeptides include those of Mycobacterium tuberculosis, which may be advantageously used as described herein given the benefits of a strong pro-inflammatory T-cell mediated immunity against tuberculosis. Additional non-limiting examples include Actinomyces polypeptides, Bacillus polypeptides, anthrax PA and LF polypeptides, Bacteroides polypeptides, Bordetella polypeptides, Bartonella polypeptides, Borrelia polypeptides, e.g., B. burgdorferi OspA, Brucella polypeptides, Campylobacter polypeptides, Capnocytophaga polypeptides, Chlamydia polypeptides, Clostridium polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides, H. influenzae type b outer membrane protein, Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides, Proteus polypeptides, Pseudomonas polypeptides, Rickettsia polypeptides, Rochalimaea polypeptides, Salmonella polypeptides, Shigella polypeptides, Staphylococcus polypeptides, Streptococcus polypeptides, S. pyogenes M proteins, Treponema polypeptides, Yersinia polypeptides, and Y. pestis Fl and V antigens.

Non-limiting examples of fungal immunogenic and antigenic polypeptides include Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Non-limiting examples of protozoan parasite immunogenic and antigenic polypeptides include those of Plasmodium falciparum, which may be advantageously used as described herein given the benefits of a strong pro-inflammatory T-cell mediated immunity against malaria. Additional non-limiting examples include Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides, P. falciparum circumsporozoite (PfCSP), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Non-limiting examples of helminth parasite immunogenic and antigenic polypeptides include Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyrne polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides.

Non-limiting examples of ectoparasite immunogenic and antigenic polypeptides include polypeptides from fleas; ticks, including hard ticks and soft ticks, flies, such as midges, mosquitos, sand flies, black flies, horse flies, horn flies, deer flies, tsetse flies, stable flies, myiasis-causing flies and biting gnats; ants; spiders, lice; mites; and true bugs, such as bed bugs and kissing bugs.

Non-limiting examples of cancer or tumor-associated antigenic polypeptides include tumor-specific immunoglobulin variable regions, GM2, Tn, sTn, Thompson-Friedenreich antigen (TF), Globo H, Le(y), MUC1, MUC2, MUC3, MUC4, MUC5AC, MUC5B, MUC7, carcinoembryonic antigens, beta chain of human chorionic gonadotropin (hCG beta), HER2/neu, PSMA, EGFRvIII, KSA, PSA, PSCA, GP100, MAGE 1, MAGE 2, TRP 1, TRP 2, tyrosinase, MART-1, PAP, CEA, BAGE, MAGE, RAGE, and related proteins.

In addition to the foregoing polypeptides, additional polypeptides of the disclosure are fragments or variants of the polypeptides described herein.

Cancer or tumor-associated antigenic and immunogenic polypeptides of the disclosure may be used to prevent or treat, or cure, ameliorate, or lessen the severity of, cancer. Non-limiting examples of possible cancers to treat include cancers of oral cavity and pharynx (i.e., tongue, mouth, pharynx), digestive system (i.e., esophagus, stomach, small intestine, colon, rectum, anus, anal canal, liver, gallbladder, pancreas), respiratory system (i.e., larynx, lung), bones, joints, soft tissues (including heart), skin, melanoma, breast, reproductive organs (i.e., cervix, endometrium, ovary, vulva, vagina, prostate, testis, penis), urinary system (i.e., urinary bladder, kidney, ureter, and other urinary organs), eye, brain, endocrine system (i.e., thyroid and other endocrine), lymphoma (i.e., Hodgkin's disease, non-Hodgkin's lymphoma), multiple myeloma, leukemia (i.e., acute lymphocytic leukemia, chronic lymphocytic leukemia, acute myeloid leukemia, chronic myeloid leukemia).

Methods of Use

The disclosure includes therapeutic methods, such as a method of producing an immune response, in a human or animal subject, to an antigen by administering nucleic acid molecule(s) as described herein. In some embodiments, the method comprises administering a composition comprising the molecule(s). The composition may be a pharmaceutical composition or formulation as described herein. The immune response may be any action by the immune system of a treated subject that is due to the administered molecule(s). In mammals, an immune response includes both cellular activities and the activities of soluble molecules, such as cytokines and antibodies.

In other embodiments, the method increases IFN-β, or cytokine (or chemokines), levels or production in a cell, optionally an in vivo cell within a subject. In some cases, the increased production is of a pro-inflammatory cytokine, with IL-6, IP-10, IL-1, IL-12, and RANTES as non-limiting examples. In further embodiments, increased IFN-β production in a subject may advantageously result in the activation of dendritic cells (DCs), enhance antibody responses, and/or enhance CD8+ responses induced by cross priming. Additional embodiments include treating a cell ex vivo and then re-introducing the cells into the subject from whom or which they came.

In further embodiments, the method further comprise contacting a cell expressing an agonist of the RIG-I pathway with interferon-α (IFN-α) and/or tumor necrosis factor alpha (TNF-α). In embodiments of these methods wherein the cell is in vivo, the IFN-α and/or TNF-α may be administered to the subject separately, or together with, the nucleic acid molecule encoding the agonist. Additional embodiments include providing the cell with a nucleic acid molecule which expresses IFN-α and/or TNF-α.

A range of cells may be used as part of a method described herein. Embodiments of the disclosed methods of course include cells that have a RIG-I pathway subject as described herein, or that are subject to the effects of a disclosed agonist in increasing IFN-β levels or production. Non-limiting examples include dendritic cells, such as plasmacytoid dendritic cells, fibroblasts, and hematopoietic cells.

A therapeutic method of the disclosure includes administering nucleic acid molecule(s) by any convenient, appropriate, or desirable means. The administration may be to immunize a human or animal subject via delivery and expression of the described antigen. This may be readily achieved via administration of an amount sufficient to generate an immune response to the antigen. Non-limiting examples include administration by intramuscular, subcutaneous, intraarterial, intracapsular, intraorbital, intracardiac, intradermal, intramedullary, intraspinal, intravenous, intraperitoneal, intranasal, intraocular, intrathecal, intraventricular, intravesicular, intratracheal, transdermal, transmucosal (i.e., across a mucous membrane), subcuticular, subcapsular, subarachnoid, or transtracheal injection.

Additional routes of administration include oral, buccal, sublingual, rectal, transdermal or intradermal, vaginal, transmucosal or mucosal, nasal, intestinal, or parenteral delivery. Administration may also be to a body cavity, such as, but not limited to, the lung, mouth, nasal cavity, stomach, peritoneum, intestine, heart chamber, vein, artery, capillary, lymphatic, uterus, vagina, rectum, and ocular cavity.

Administration to a particular tissue or organ, such as the brain or a bladder of an animal or human subject, may also be used. Other non-limiting examples include administration or delivery to muscle, skin, lung, liver, spleen, bone marrow, thymus, heart, lymph, blood, bone, cartilage, mucosal tissue, pancreas, kidney, gall bladder, stomach, intestine, testis, ovary, uterus, vaginal tissue, rectum, nervous system, eye, gland, tongue and connective tissue. In some embodiments, administration may be systemic or peripheral to a subject such that the administered molecule(s) or composition(s), other than to the subject's nervous system, enters the subjects system and subsequently undergoes metabolism in the subject.

In other embodiments, administration may be by microprojectile bombardment (MB) of a nucleic acid molecule of the disclosure or a composition thereof. Methods comprising MB frequently include the use of a bolistic (or “gene gun”) injector or device as known to the skilled person, and successful introduction of nucleic acids have been repeatedly reported (see for example, U.S. Pat. Nos. 5,538,880; 5,550,318; and 5,610,042; as well as WO 94/09699). Other non-limiting examples include particle accelerators like Med-E-Jet (Vahlsing, H., et al., J. Immunol. Methods 171, 11-22 (1994)), Pigjet (Schrijver, R., et al., Vaccine 15, 1908-1916 (1997)), Biojector (Davis, H., et al., Vaccine 12, 1503-1509 (1994); Gramzinski, R., et al., Mol. Med. 4, 109-118 (1998)), AdvantaJet, or Medijector. In some embodiments, nucleic acid molecule(s) are introduced into skin cells via MB.

In some embodiments, particles or beads as described herein are coated with more than one copy of nucleic acid molecule(s) and then delivered into target cells, such as those of an animal or human subject by a propelling force. Non-limiting examples of materials used in, or on, a particle or bead include tungsten, platinum, or gold. While a particle or bead may be coated with nucleic acid molecule(s) by their precipitation onto metal, such coating is optional because a particle or bead may contain the molecules rather than be coated with it.

In further embodiments, administration may be by catheter infusion, gelfoam sponge depots (or other depot materials like hydrojels), transdermal patches, needle arrays, needle-free injection, needle-free devices, osmotic pumps, hydrodynamic delivery, tablet or pill formulations, and topical application, such as skin creams, polynucleotide coated suture(s) (Qin et al., Life Sciences 65, 2193-2203 (1999)) or application during surgery.

In further embodiments, the administration may be in combination with one or more adjuvants as known in the field of immunization protocols. Because the effects of an adjuvant is not antigen-specific, a variety of adjuvants may be used in the disclosed methods. Non-limiting examples of adjuvants include alum; a bacterial molecule, such as muramyl dipeptide (N-acetylmuramyl-L-alanyl-D-isoglutamine or MDP) or a derivative thereof (such as the amino acid derivative threonyl-MDP or the fatty acid derivative MTPPE), or a peptidoglycan; an alkyl lysophosphilipid (ALP); BCG (bacillus Calmette-Guerin, an attenuated strain of Mycobacterium) or BCG-cell wall skeleton (CWS); a teichoic acid from Gram negative cells, such as a lipoteichoic acid (LTA), a ribitol teichoic acid (RTA), and a glycerol teichoic acid (GTA); a hemocyanins or hemoerythrin, such as keyhole limpet hemocyanin (KLH), an arthropod hemocyanin, or an arthropod hemoerythrin; a polysaccharide adjuvant, such as a pneumococcal polysaccharide, chitin, chitosan, or deacetylated chitin; trehalose dimycolate; an amphipathic and surface active agent, such as saponin or a derivative thereof like QS21 (Cambridge Biotech); Quil A; lentinen; MF-59 (or MF59); MPLA; or a detoxified endotoxin, such as that disclosed in U.S. Pat. No. 4,866,034. An additional non-limiting example is Ribi's Adjuvant System (RAS). Additional non-limiting examples of adjuvants are described by Vogel F R, et al. (“A compendium of vaccine adjuvants and excipients.” Pharm Biotechnol. 1995, 6:141-228), which is incorporated herein by reference as if fully set forth. The disclosed compositions and methods may include use of any one or more of the adjuvants described therein.

The methods of the disclosure optionally comprise multiple administrations of the disclosed nucleic acid molecule(s) or a composition or formulation thereof. In some embodiments, the number of repeat administrations is less than six, less than four, or about one, two, or three. The administrations may be spaced by various time intervals. Non-limiting examples include an interval from about two to about twelve weeks, about four to about six weeks, or about eight to about ten weeks. In additional embodiments, it may be advantageous to rapidly administer vaccine, in which case the intervals may be shortened to be about 1 day, about 2 days, about 3 days, about 4 days, about 5 days, about 6 days, about 7 days, about 8 days, about 9 days, about 10 days, about 11 days, about 12 days, or about 13 days. Booster administrations after an interval of about 1, about 2, about 3, about 4, or about 5 or more years may also be used.

The course of the induced, promoted, or generated immune response may be followed by assays for antibodies or immune cells against the antigen expressed by the administered molecule(s). The assays may be performed by use of an antibody or cell containing fluid from a treated subject. Non-limiting examples include serum, plasma, or blood from a subject. Representative assay techniques are disclosed in U.S. Pat. Nos. 3,791,932; 3,949,064 and 4,174,384. Assays for other immune responses can also be performed. In some embodiments, an assay measures an immunological surrogate of protection (such as instance, levels of serum antibodies against HBV, influenza, or measles as non-limiting examples) from challenge with a pathogen expressing the antigen.

As noted above, the molecule(s) of the disclosure may be administered via a pharmaceutical composition. A pharmaceutical composition for administration according to the methods of the disclosure may be formulated according to known methods, such as by combination of the nucleic acid molecule(s) of the disclosure with a pharmaceutically acceptable carrier or vehicle as described herein. Well known vehicles and their preparation are described, for example, in Remington's Pharmaceutical Sciences, 16th Ed., A. Osol, ed., Mack Publishing Co., Easton, Pa. (1980), and Remington's Pharmaceutical Sciences, 19th Ed., A. R. Gennaro, ed., Mack Publishing Co., Easton, Pa. (1995).

Non-limiting examples of a pharmaceutical composition include formulation that is an emulsion, gel, solution, suspension, lyophilized form, or any other suitable form for administration as known to the skilled person. A pharmaceutical composition may also contain pharmaceutically acceptable additives including, for example, diluents, binders, stabilizers, and preservatives. In some embodiments, pharmaceutically acceptable salts of the nucleic acid molecules described herein are prepared for use from acceptable non-toxic bases including organic bases (primary, secondary, and tertiary amines, basic amino acids, etc.) and inorganic bases (sodium, potassium, lithium, ammonium, calcium, magnesium, etc.).

In some embodiments, a first coding region encoding a RIG-I pathway agonist and a second coding region encoding an antigen are administered via a single molecule. In other embodiments, the two coding regions are contained on separate vectors which may be optionally administered via a single composition containing them. Alternatively, the two separate vectors may be separately administered to a subject simultaneously or with a time interval between them.

An effective amount of the molecule(s) to administer depends upon a number of factors including, as non-limiting examples, the biological activity of the encoded agonist and antigen; the strength of the promoter(s) used; the age and weight of the subject; and the route of administration. The precise amount, number of doses, and timing of doses can be readily determined by those skilled in the art.

Kits

The disclosure also provides kits for use in delivering a nucleic acid containing composition to a subject of the disclosure. Each kit includes a container holding a sufficient amount of nucleic acid therapeutic molecule(s) encoding an agonist and an antigen as described herein. Optionally, each kit includes, in the same or in a different container, an adjuvant composition for use with the therapeutic molecule(s). Any of the components of the kits can be provided in a single container or in multiple containers. Non-limiting examples of containers include glass containers, plastic containers, or strips of plastic or paper.

In some embodiments, the kit includes from about 1 ng to about 30 mg of the therapeutic molecule(s), such as from about 100 ng to about 10 mg of the molecule(s). The molecule(s), or composition comprising them, may be included as a liquid solution or they may be included in lyophilized form, such as in the form of a dried powder or a cake. If included in lyophilized form, the dried material may also include any salts, entry enhancing agents, transfection facilitating agents, and additives of the composition in dried form. The kit may further comprise a container with sterile pyrogen-free water for reconstitution or hydration of the lyophilized material before use.

A kit with the molecule(s) may comprise a hermetically sealed container enclosing an amount of the liquid solution or lyophilized material containing the molecule(s) suitable for an effective dose thereof, or multiples of an effective dose. In additional embodiments, a kit may further comprise an administration means. Non-limiting examples include syringes and needles, catheters, biolistic injectors, particle accelerators (e.g. “gene guns” or pneumatic “needleless” injectors), gelfoam sponge depots, other commercially available depot materials (e.g. hydrojels), and decanting or topical applications during surgery.

In further embodiments, a kit can also comprise an instruction sheet for use of kit components in a method as described herein.

Having now provided a written description, the same will be more readily understood through reference to the following examples which are provided by way of illustration, and are not intended to be limiting of the disclosure, unless specified.

### EXAMPLES

### Results

### Activation of IFN-β Response in Transfected Mouse Cells by RNA

Previous studies have shown that RIG-I/MDA-5 is an essential detector of RNA viruses including Newcastle disease virus, Sendai virus, Vesicular stomatitis virus, influenza virus, measles virus, hepatitis C virus and Japanese encephalitis virus (Berghall et al., 2006; Chang et al., 2006; Kato et al., 2006; Melchjorsen et al., 2005). Furthermore, Plumet et al (Plumet et al., 2007) has shown that viral leader sequences from measles and vesicular stomatitis virus can activate production of IFN-β through RIG-I pathway in transfected human liver epithelial and kidney cells. Therefore, we tested whether these viral leader RNAs may activate IFN-β response in mouse cell lines in vitro. RNA was transcribed in vitro using a T7 promoter system which produces RNA with a 5′ triphosphate that has been reported to be important for activation of RIG-I (Hornung et al., 2006; Plumet et al., 2007). Transfection was done in L929 mouse fibroblast cell line, which has previously been shown to have a functional RIG-I pathway (Yoneyama et al., 2004), and VM92 mouse melanoma cells, which showed elevated IFN-β expression when transfected with a plasmid DNA encoding the N-terminal tandem CARD domains of RIG-I (ΔRIG-I). ΔRIG-I lacks the helicase domain and functions as a constitutive active protein for positively signaling the RIG-I pathway for activation of the type I IFN response (Yoneyama et al., 2004). The increased expression of IFN-β in response to ΔRIG-I, but not to the backbone vector pDNA, suggested the presence of a functional RIG-I pathway in VM92 cells (FIG. 1A). Upon transfection of RNA into L929 or VM92 cells, both measles virus leader (ML) and vesicular stomatitis virus leader (VSL) RNA showed significant activation in IFN-β expression in the two cell lines (FIGS. 1 B and C). This activation of IFN-β production was greater than in cells transfected with poly I:C, a synthetic dsRNA analogue known as agonist for TLR3 and MDA-5 (Alexopoulou et al., 2001; Gitlin et al., 2006; Kato et al., 2006). However, RNA transcripts from the hepatitis C virus untranslated region and the adenoviral VAI and VAII had no effect on IFN-β response (FIG. 1 & data not shown), indicating that not all dsRNAs were able to activate IFN-β production in vitro. The expression of chemokine IP-10, which has been shown to be associated with active RIG-I pathway (Yoneyama et al., 2004), was also upregulated in cells transfected with poly I:C, ML and VSL RNA (FIG. 1D).

### Activation of RIG-I Pathway in Mouse Muscle Upregulates Cytokine Expression

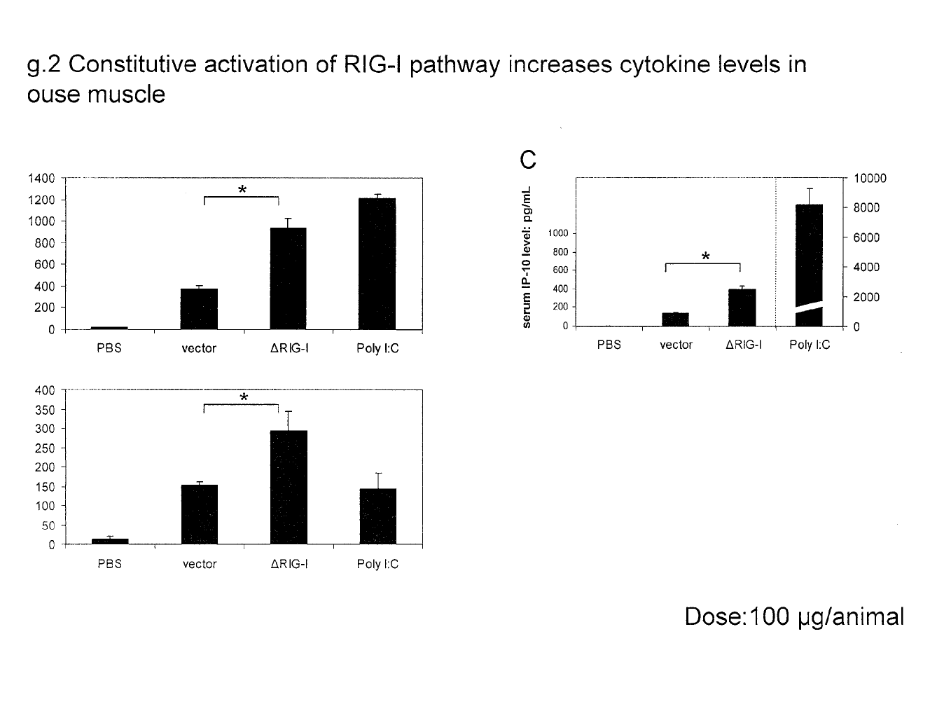

Vaccines are commonly delivered by intramuscular injections. However, whether the RIG-I pathway is active in the skeletal muscle has not been established. We therefore tested whether the cytokine expression levels are upregulated in mouse muscles after injection with a plasmid DNA encoding a constitutive active RIG-I (ΔRIG-I). ΔRIG-I injection increased IP-10 and IL-1β levels in muscle 6 h post-injection (FIG. 2). Furthermore, serum IP-10 levels were also elevated 6 h post-injection. These data suggest that activation of RIG-I pathway in mouse skeletal muscle results in elevated cytokine levels in the injected tissue as well as in the serum.

### ML RNA Induced Expression of Cytokines in Mouse Muscle

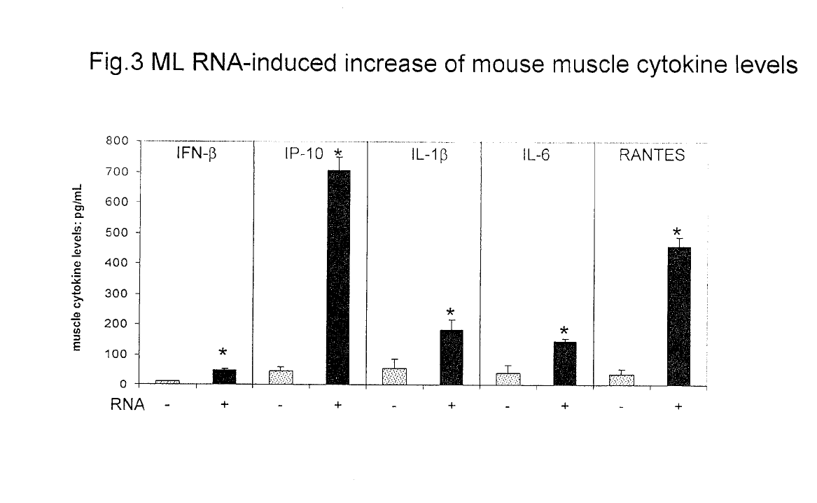

To test whether in vitro synthesized ML RNA which greatly enhanced IFN-β level in transfected cell lines can also enhance cytokine levels in mouse muscle, we injected 100 μg of ML RNA into mouse muscle and analyzed various cytokine levels 6 h post-injection. ML RNA injections increased IFN-β levels 4.5 fold compared to injections of PBS vehicle alone. Various pro-inflammatory cytokines were also enhanced with ML RNA injection in muscle: IP-10, 15 fold; IL-1β, 3 fold; IL-6, 3 fold; and RANTES, 13 fold (FIG. 3). Moreover, serum IP-10 level was 7 fold higher after ML RNA injection compared to PBS (data not shown). These data suggested that the intramuscular injection of ML RNA can stimulate IFN-β and cytokine/chemokine expression. Furthermore, since increased expression of the selected cytokines have all been linked to activation of RIG-I pathway (Kato et al., 2006; Kubota et al., 2006; Yoneyama et al., 2004; Yoshida et al., 2007), these results suggest that ML RNA may increase cytokine production in muscle tissue by activating the RIG-I pathway.

ML RNA Induced Enhancement of Antigen Specific Antibody Titers with Protein Vaccine

To assess whether ML RNA can function as a molecular adjuvant to enhance antigen-specific humoral immune responses to protein vaccines, we injected the trivalent influenza vaccine Fluzone® (TIV) in the presence of 100 μg of ML RNA into mouse muscle and analyzed the antibody-specific titer. Serum collected from mice injected with TIV and ML RNA showed a 2 to 3 fold enhancement in anti-TIV titers compared to mice injected with TIV alone. This enhancement was observed from serum sample collected 20 days after initial injection, and was sustained through day 33. The results suggest that ML RNA may function as a molecular adjuvant in enhancing antibody-specific titers of protein vaccine.

### Materials & Methods

### Plasmids

pBAD.T7HCV, pBAD.T7ML and pBAD.T7VSL containing T7 promoter encoding hepatitis C virus 5′ untranslated region (Sumpter et al., 2005), measles virus leader sequence (Plumet et al., 2007) and vesicular stomatitus virus leader sequence (Gupta et al., 1998) were cloned into the pBAD-TOPO vector (Invitrogen, Carlsbad, Calif.). pAdVAntage™ Vector encoding adenoviral virus-associated RNA (VAI and VAII) was purchased from Promega. Plasmid VR9033 encodes the N-terminal tandem CARD domains of RIG-I (amino acids 1-264) was synthesized (Blue Heron Bio, Bothell, Wash.) and cloned into the EcoRI/BamHI site of VR10551 (Vical Inc., San Diego, Calif.) expression vector.

### RNA Synthesis and Purification

RNA transcription templates were obtained by PCR using the pBAD.T7HCV, pBAD.T7ML and pBAD.T7VSL to generate a precise 3′ end. RNA synthesis templates for adenoviral VAI and VAII RNA were obtained by PCR with primers encoding the T7 promoter sequence. In vitro RNA transcription was done using MEGAshortscript™ T7 Kit (Ambion, Austin, Tex.) and purified with mirVana™ miRNA Isolation Kit (Ambion, Austin, Tex.) following manufacturer's protocol.

### Cell Lines, Transfection and ELISA Assay

L929 cells were acquired from ATCC and maintained in Eagle's Minimum Essential medium with 5% fetal bovine serum and transfected in 24-well plates with Lipofectin (Invitrogen, Carlsbad, Calif.) following manufacturer's protocol. Murine cell lines, such as VM 92 cells (Vical Inc., San Diego, Calif.) were maintained in RPMI with 10% fetal bovine serum and transfected with DMRIE:DOPE as previously described (Jimenez et al., 2007). Cell culture supernatants were collected 48 h post-transfection and analyzed for IFN-β and IP-10 level using ELISA kits (R&D systems, Minneapolis, Minn.).

### In Vivo Mouse Studies

Female BALB/c mice (6-8 weeks old) received bilateral intramuscular injections in Rectus femoris with 50 μl of the indicated samples per site. Animal procedures and husbandry were compliant with the ‘Guide for the Use and Care of Laboratory Animals’(National Academy Press, Washington, D.C., 1996), and were additionally subject to approval by the Institutional Animal Care and Use Committee. For analysis of cytokine production, sera or muscle tissues were collected 6 h post-injection. Muscle tissues were processed as previously described (Hartikka et al., 1996) and assayed for IFN-β, IP-10, IL-1β, IL-6 and RANTES production using ELISA kits (R&D systems, Minneapolis, Minn.). For measurement of antigen specific antibody responses, mice were given a second injection 3 weeks after initial injection. Sera were collected 5 weeks after initial injection to test for antigen-specific antibody responses.

### Anti-TIV ELISA

Plates were coated with Fluzone® (0.9 μg/mL of HA, Sanofi Pasteur Inc., Swiftwater, Pa.) in borate-buffered saline (BBS, 89 mM boric acids, 90 mM Nacl, pH 8.3). The plates were stored overnight at 4° C. and the wells washed four times with BBST (BBS supplemented with 0.05% Tween 20, vol:vol). The wells were then incubated for 1 hr with BB (BBS supplemented with 1% BSA, wt:vol) and washed twice with BBST again. Twofold serial dilutions of mouse serum in BB, starting at 1:50, were made in successive wells and the solutions were incubated for 2 h at room temperature. Wells were then rinsed four times with BBST. For detection, alkaline phosphatase conjugated goat anti-mouse IgG-Fc (Jackson ImmunoResearch Laboratories, Cat. No. 115-055-008, West Grove, Pa.) diluted 1:5000 in BBS was added at 50 μl/well and the plates were incubated at room temperature for 2 h. After four washings in BBST, 50 μl of substrate (1 mg/ml p-nitrophenyl phosphate, Calbiochem, San Diego, Calif., in 50 mM sodium bicarbonate buffer, pH 9.8 and 1 mM MgCl₂) was incubated for 90 min at room temperature and absorbance readings were performed at 405 nm. The titer of the sera was determined by using the reciprocal of the last dilution yielding an absorbance twice above background, established using pre-immune serum diluted 1:20.

### Example A

### Materials and Methods

A model antigen used is the influenza A M2 protein, which contains CD4 T cell, CD8 T cell, and B cell epitopes. Standard immunological assays measure T and B cell responses to the antigen and an increase or enhancement in immune responses compared to expression of the M2 protein alone. The M2 protein is expressed via its coding region under the control of a RNA Pol II promoter (such as a cytomegalovirus, CMV, promoter) and in a suitable vector.

A first RIG-I protein agonist is a truncated form of RIG-I protein (designated ΔRIG-I) containing the 2 CARDS within the N-terminal 264 amino acids (see Yoneyama, Nat Imm 2004). A second agonist is a stimulatory dsRNA species encoded by a pDNA (plasmid DNA) and expressed under the control of a RNA Pol III promoter.

In some experiments, the stimulatory dsRNA RIG-I agonist is cis relative to the M2 coding region and so on the same nucleic acid molecule or vector.

### Example B

### ΔRIG-I as RIG-I Agonist

A pDNA which co-expresses influenza M2 and ΔRIG-I are introduced into human and mouse cell lines. The levels of IFN-β, M2, and possibly ΔRIG-I production are measured or detected after pDNA transfection. Decreases in M2 expression may occur and their effects on IFN-β production will be observed and recorded.

Additionally, the pDNA is administered (via intramuscular, IM, or intradermal, ID, means) to mice. The immunized mice are observed for an increase or enhancement of the immune response to M2 and/or protection against influenza virus lethal challenge in comparison to control mice selected from untreated, immunized with ΔRIG-I negative pDNA (expressing M2), and immunized with M2 negative pDNA (expressing ΔRIG-I).

The possible enhancements to the immune response may entail a “dose sparing” effect observed for both antibody and T cell responses and/or a higher magnitude immune response observed at “plateau” pDNA doses. In other words, the maximum immune response observed with the antigen alone will be significantly increased in the presence of a RIG-I agonist.

### Example C

### dsRNA as RIG-I Agonist Produced as a Separate Transcript

A pDNA which co-expresses influenza M2 under the control of a CMV promoter and a dsRNA species selected from the 5′ or 3′ UTR of hepatitis C virus (HCV) under the control of a RNA Pol III promoter is used in cell lines and mice as described in Example 2.

### Example D

### dsRNA as RIG-I Agonist in Cis with M2 mRNA

The 5′ or 3′ UTR of HCV is placed 5′, or 3′, relative to the M2 coding region to permit transcription of an mRNA comprising both. The effect of the UTR sequences on IFN-β production is tested in vitro and observed and recorded relative to IFN-β production and M2 expression. A pDNA containing the construct is also used in cell lines and mice as described in Example 2.

### Example E

### Exemplary Expression Constructs

Arrangement of elements of exemplary expression constructs used in the above Examples and within the scope of the disclosure are listed below. CMV refers to the CMV promoter; M2 refers to the antigen coding region; IRES refers to internal ribosome entry site; ΔRIG-I refers to the ΔRIG-I coding region; RBGterm refers to rabbit beta globin terminator; “stop” refers to premature termination of translation to produce a truncated, non-active polypeptide; Pol III refers to a Pol III promoter; and polyTterm refers to a polyT tail terminator. The fourth construct is VR4759 as discussed above.

1) CMV-M2-IRES-ΔRIG-I-RBGterm

2) CMV-M2-IRES-ΔRIG-Istop-RBGterm

3) CMV-M2-RBGterm-Pol III-dsRNA (HCV 5′ UTR)-polyTterm

4) CMV-M2-RBGterm

5) CMV-ΔRIG-I-RGBterm

All references cited herein, including patents, patent applications, and publications, are hereby incorporated by reference in their entireties, whether previously specifically incorporated or not.

Having now fully provided the instant disclosure, it will be appreciated by those skilled in the art that the same can be performed within a wide range of equivalent parameters, concentrations, and conditions without departing from the spirit and scope of the disclosure and without undue experimentation.

While the disclosure has been described in connection with specific embodiments thereof, it will be understood that it is capable of further modifications. This application is intended to cover any variations, uses, or adaptations of the disclosure following, in general, the disclosed principles and including such departures from the disclosure as come within known or customary practice within the art to which the disclosure pertains and as may be applied to the essential features hereinbefore set forth.

### REFERENCES

Akira, S., and Takeda, K. (2004). Toll-like receptor signalling. Nat Rev Immunol 4, 499-511.
Alexopoulou, L., Holt, A. C., Medzhitov, R., and Flavell, R. A. (2001). Recognition of double-stranded RNA and activation of NF-kappaB by Toll-like receptor 3. Nature 413, 732-738.
Berghall, H., Siren, J., Sarkar, D., Julkunen, I., Fisher, P. B., Vainionpaa, R., and Matikainen, S. (2006). The interferon-inducible RNA helicase, mda-5, is involved in measles virus-induced expression of antiviral cytokines. Microbes Infect 8, 2138-2144.
Bredow, S., Kleinert, H., and Benecke, B. J. (1990). Sequence and factor requirements for faithful in vitro transcription of human 7SL DNA. Gene 86, 217-225.
Chang, T. H., Liao, C. L., and Lin, Y. L. (2006). Flavivirus induces interferon-beta gene expression through a pathway involving RIG-1-dependent IRF-3 and PI3K-dependent NF-kappaB activation. Microbes Infect 8, 157-171.
Chu, W. M., Liu, W. M., and Schmid, C. W. (1995). RNA polymerase III promoter and terminator elements affect Alu RNA expression. Nucleic Acids Res 23, 1750-1757.
Curtsinger, J. M., Lins, D. C., Johnson, C. M., and Mescher, M. F. (2005). Signal 3 tolerant CD8 T cells degranulate in response to antigen but lack granzyme B to mediate cytolysis. J Immunol 175, 4392-4399.
Davis, H. L., Michel, M. L., Mancini, M., Schleef, M., and Whalen, R. G. (1994). Direct gene transfer in skeletal muscle: plasmid DNA-based immunization against the hepatitis B virus surface antigen. Vaccine 12, 1503-1509.
Day, P. J., Flora, P. S., Fox, J. E., and Walker, M. R. (1991). Immobilization of polynucleotides on magnetic particles. Factors influencing hybridization efficiency. Biochem J 278 (Pt 3), 735-740.
Egan, M., and Israel, Z. (2002). The use of cytokines and chemokines as genetic adjuvants for plasmid DNA vaccines. Clinical and Applied Immunology Reviews 2, 255-287.
Gitlin, L., Barchet, W., Gilfillan, S., Cella, M., Beutler, B., Flavell, R. A., Diamond, M. S., and Colonna, M. (2006). Essential role of mda-5 in type I IFN responses to polyriboinosinic:polyribocytidylic acid and encephalomyocarditis picornavirus. Proc Natl Acad Sci USA 103, 8459-8464.
Goeddel, D. V. (1990). Systems for heterologous gene expression. Methods Enzymol 185, 3-7.
Gramzinski, R. A., Millan, C. L., Obaldia, N., Hoffman, S. L., and Davis, H. L. (1998). Immune response to a hepatitis B DNA vaccine in Aotus monkeys: a comparison of vaccine formulation, route, and method of administration. Mol Med 4, 109-118.
Gupta, A. K., Drazba, J. A., and Banerjee, A. K. (1998). Specific interaction of heterogeneous nuclear ribonucleoprotein particle U with the leader RNA sequence of vesicular stomatitis virus. J Virol 72, 8532-8540.
Hartikka, J., Sawdey, M., Cornefert-Jensen, F., Margalith, M., Barnhart, K., Nolasco, M., Vahlsing, H. L., Meek, J., Marquet, M., Hobart, P., et al. (1996). An improved plasmid DNA expression vector for direct injection into skeletal muscle. Hum Gene Ther 7, 1205-1217.
Hertzog, P. J., O'Neill, L. A., and Hamilton, J. A. (2003). The interferon in TLR signaling: more than just antiviral. Trends Immunol 24, 534-539.
Hornung, V., Ellegast, J., Kim, S., Brzozka, K., Jung, A., Kato, H., Poeck, H., Akira, S., Conzelmann, K. K., Schlee, M., et al. (2006). 5′-Triphosphate RNA is the ligand for RIG-I. Science 314, 994-997.
Jimenez, G. S., Planchon, R., Wei, Q., Rusalov, D., Geall, A., Enas, J., Lalor, P., Leamy, V., Vahle, R., Luke, C. J., et al. (2007). Vaxfectin-formulated influenza DNA vaccines encoding NP and M2 viral proteins protect mice against lethal viral challenge. Hum Vaccin 3, 157-164.
Jopling, C. L., Yi, M., Lancaster, A. M., Lemon, S. M., and Sarnow, P. (2005). Modulation of hepatitis C virus RNA abundance by a liver-specific MicroRNA. Science 309, 1577-1581.
Kato, H., Sato, S., Yoneyama, M., Yamamoto, M., Uematsu, S., Matsui, K., Tsujimura, T., Takeda, K., Fujita, T., Takeuchi, O., and Akira, S. (2005). Cell type-specific involvement of RIG-I in antiviral response. Immunity 23, 19-28.
Kato, H., Takeuchi, O., Sato, S., Yoneyama, M., Yamamoto, M., Matsui, K., Uematsu, S., Jung, A., Kawai, T., Ishii, K. J., et al. (2006). Differential roles of MDA5 and RIG-I helicases in the recognition of RNA viruses. Nature 441, 101-105.
Kawai, T., Takahashi, K., Sato, S., Coban, C., Kumar, H., Kato, H., Ishii, K. J., Takeuchi, O., and Akira, S. (2005). IPS-1, an adaptor triggering RIG-1- and MdaS-mediated type I interferon induction. Nat Immunol 6, 981-988.
Kleinert, H., Gladen, A., Geisler, M., and Benecke, B. J. (1988). Differential regulation of transcription of human 7 S K and 7 S L RNA genes. J Biol Chem 263, 11511-11515.
Kodama, K., Katayama, Y., Shoji, Y., and Nakashima, H. (2006). The features and shortcomings for gene delivery of current non-viral carriers. Curr Med Chem 13, 2155-2161.
Kubota, K., Sakaki, H., Imaizumi, T., Nakagawa, H., Kusumi, A., Kobayashi, W., Satoh, K., and Kimura, H. (2006). Retinoic acid-inducible gene-I is induced in gingival fibroblasts by lipopolysaccharide or poly IC: possible roles in interleukin-1beta, -6 and -8 expression. Oral Microbiol Immunol 21, 399-406.
Le Bon, A., Etchart, N., Rossmann, C., Ashton, M., Hou, S., Gewert, D., Borrow, P., and Tough, D. F. (2003). Cross-priming of CD8+ T cells stimulated by virus-induced type I interferon. Nat Immunol 4, 1009-1015.
Le Bon, A., Schiavoni, G., D'Agostino, G., Gresser, I., Belardelli, F., and Tough, D. F. (2001). Type i interferons potently enhance humoral immunity and can promote isotype switching by stimulating dendritic cells in vivo. Immunity 14, 461-470.
Luft, T., Pang, K. C., Thomas, E., Hertzog, P., Hart, D. N., Trapani, J., and Cebon, J. (1998). Type I IFNs enhance the terminal differentiation of dendritic cells. J Immunol 161, 1947-1953.
Marrack, P., Kappler, J., and Mitchell, T. (1999). Type I interferons keep activated T cells alive. J Exp Med 189, 521-530.
Melchjorsen, J., Jensen, S. B., Malmgaard, L., Rasmussen, S. B., Weber, F., Bowie, A. G., Matikainen, S., and Paludan, S. R. (2005). Activation of innate defense against a paramyxovirus is mediated by RIG-I and TLR7 and TLR8 in a cell-type-specific manner. J Virol 79, 12944-12951.
Meylan, E., Curran, J., Hofmann, K., Moradpour, D., Binder, M., Bartenschlager, R., and Tschopp, J. (2005). Cardif is an adaptor protein in the RIG-I antiviral pathway and is targeted by hepatitis C virus. Nature 437, 1167-1172.
Nguyen, K. B., Watford, W. T., Salomon, R., Hofmann, S. R., Pien, G. C., Morinobu, A., Gadina, M., O'Shea, J. J., and Biron, C. A. (2002). Critical role for STAT4 activation by type 1 interferons in the interferon-gamma response to viral infection. Science 297, 2063-2066.
Pasare, C., and Medzhitov, R. (2003). Toll pathway-dependent blockade of CD4+CD25+ T cell-mediated suppression by dendritic cells. Science 299, 1033-1036.
Pedersen, N., Hansen, S., Heydenreich, A. V., Kristensen, H. G., and Poulsen, H. S. (2006). Solid lipid nanoparticles can effectively bind DNA, streptavidin and biotinylated ligands. Eur J Pharm Biopharm 62, 155-162.
Plumet, S., Herschke, F., Bourhis, J. M., Valentin, H., Longhi, S., and Gerlier, D. (2007). Cytosolic 5′-triphosphate ended viral leader transcript of measles virus as activator of the RIG I-mediated interferon response. PLoS ONE 2, e279.
Proietti, E., Bracci, L., Puzelli, S., Di Pucchio, T., Sestili, P., De Vincenzi, E., Venditti, M., Capone, I., Seif, I., De Maeyer, E., et al. (2002). Type I IFN as a natural adjuvant for a protective immune response: lessons from the influenza vaccine model. J Immunol 169, 375-383.
Prow, T., Grebe, R., Merges, C., Smith, J. N., McLeod, D. S., Leary, J. F., and Lutty, G. A. (2006). Nanoparticle tethered antioxidant response element as a biosensor for oxygen induced toxicity in retinal endothelial cells. Mol V is 12, 616-625.
Qin, Y. J., Zhang, J. F., Wei, Y. J., Ding, J. F., Chen, K. H., and Tang, J. (1999). Gene suture—a novel method for intramuscular gene transfer and its application in hypertension therapy. Life Sci 65, 2193-2203.
Schnare, M., Barton, G. M., Holt, A. C., Takeda, K., Akira, S., and Medzhitov, R. (2001). Toll-like receptors control activation of adaptive immune responses. Nat Immunol 2, 947-950.
Schrijver, R. S., Langedijk, J. P., Keil, G. M., Middel, W. G., Maris-Veldhuis, M., Van Oirschot, J. T., and Rijsewijk, F. A. (1997). Immunization of cattle with a BHV1 vector vaccine or a DNA vaccine both coding for the G protein of BRSV. Vaccine 15, 1908-1916.
Seth, R. B., Sun, L., Ea, C. K., and Chen, Z. J. (2005). Identification and characterization of MAVS, a mitochondrial antiviral signaling protein that activates NF-kappaB and IRF 3. Cell 122, 669-682.
Sumpter, R., Jr., Loo, Y. M., Foy, E., Li, K., Yoneyama, M., Fujita, T., Lemon, S. M., and Gale, M., Jr. (2005). Regulating intracellular antiviral defense and permissiveness to hepatitis C virus RNA replication through a cellular RNA helicase, RIG-I. J Virol 79, 2689-2699.
Tuting, T., Gambotto, A., Robbins, P. D., Storkus, W. J., and DeLeo, A. B. (1999). Co-delivery of T helper 1-biasing cytokine genes enhances the efficacy of gene gun immunization of mice: studies with the model tumor antigen beta-galactosidase and the BALB/c Meth A p53 tumor-specific antigen. Gene Ther 6, 629-636.
Vahlsing, H. L., Yankauckas, M. A., Sawdey, M., Gromkowski, S. H., and Manthorpe, M. (1994). Immunization with plasmid DNA using a pneumatic gun. J Immunol Methods 175, 11-22.
Vogel, F. R., and Powell, M. F. (1995). A compendium of vaccine adjuvants and excipients. Pharm Biotechnol 6, 141-228.
Yoneyama, M., Kikuchi, M., Natsukawa, T., Shinobu, N., Imaizumi, T., Miyagishi, M., Taira, K., Akira, S., and Fujita, T. (2004). The RNA helicase RIG-I has an essential function in double-stranded RNA-induced innate antiviral responses. Nat Immunol 5, 730-737.
Yoshida, H., Imaizumi, T., Lee, S. J., Tanji, K., Sakaki, H., Matsumiya, T., Ishikawa, A., Taima, K., Yuzawa, E., Mori, F., et al. (2007). Retinoic acid-inducible gene-I mediates RANTES/CCL5 expression in U373MG human astrocytoma cells stimulated with double-stranded RNA. Neurosci Res 58, 199-206.
Zhang, X., Sun, S., Hwang, I., Tough, D. F., and Sprent, J. (1998). Potent and selective stimulation of memory-phenotype CD8+ T cells in vivo by IL-15. Immunity 8, 591-599.
Zwiorek, K., Kloeckner, J., Wagner, E., and Coester, C. (2005). Gelatin nanoparticles as a new and simple gene delivery system. J Pharm Pharm Sci 7, 22-28.

1. An isolated, purified, or recombinant nucleic acid molecule comprising, and capable of expressing,
a. a first coding region encoding a RIG-I pathway agonist; and
b. a second coding region encoding an antigen.

2. The molecule of claim 1 wherein said RIG-I pathway agonist is a RIG-I agonist.

3. The molecule of claim 2 wherein said agonist is an RNA molecule comprising a 5′ triphosphate moiety.

4. The molecule of claim 3 wherein said RNA is an ssRNA.

5. The molecule of claim 3 wherein said RNA is a dsRNA.

6. The molecule of claim 2 wherein said first coding region is operably linked to a RNA polymerase III, or RNA polymerase I, promoter capable of directing or regulating expression of said agonist; and/or
wherein said second coding region is operably linked to a RNA polymerase II promoter capable of directing or regulating expression of said antigen.

7. The molecule of claim 6 wherein said RNA polymerase III, or RNA polymerase I, promoter is heterologous relative to said first coding region; and/or
wherein said RNA polymerase II promoter is heterologous relative to said second coding region.

8. The molecule of claim 2 further comprising an RNA polymerase II promoter operably linked to said second coding regions.

9. The molecule of claim 2 further comprising an RNA polymerase I or III promoter operably linked to said first coding regions.

10. The molecule of claim 9 further wherein said first and second coding regions are fused.

11. The molecule of claim 1 wherein said molecule is a plasmid.

12. The molecule of claim 1 wherein said molecule is linear.

13. The molecule of claim 1 wherein said antigen is a tumor antigen, a Mycobacterium tuberculosis antigen, a Plasmodium falciparum antigen, or a viral antigen.

14. The molecule of claim 6 wherein said RNA polymerase III promoter is the 7SL RNA promoter.

15. An isolated, purified, or recombinant nucleic acid molecule comprising
an RNA polymerase III promoter operably linked to a coding region encoding an RNA agonist of the RIG-I pathway.

16. The molecule of claim 15 wherein said RIG-I pathway agonist is a RIG-I agonist.

17. The molecule of claim 16 wherein said agonist is an RNA molecule comprising a 5′ triphosphate moiety.

18. The molecule of claim 17 wherein said RNA is an ssRNA.

19. The molecule of claim 17 wherein said RNA is a dsRNA.

20. The molecule of claim 15 wherein said RNA polymerase III promoter is heterologous relative to said coding region.

21. The molecule of claim 15 wherein said molecule is a plasmid.

22. The molecule of claim 15 wherein said molecule is a linear molecule.

23. The molecule of claim 15 wherein said RNA polymerase III promoter is the 7SL RNA promoter.

24-63. (canceled)


SEQUENCE LISTING (7 sequences)

SEQ ID NO: 1 - protein, 925 aa, Homo sapiens [sequence omitted]

SEQ ID NO: 2 - protein, 540 aa, Homo sapiens [sequence omitted]

SEQ ID NO: 3 - DNA, 341 bp, Hepatitis C virus
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcacc

SEQ ID NO: 4 - RNA, 19 nt, Homo sapiens
agcuuaaccuguccuucaa

SEQ ID NO: 5 - RNA, 24 nt, Homo sapiens
ggggcugacccugaaguucaucuu

SEQ ID NO: 6 - RNA, 24 nt, Homo sapiens
ggggaugaacuucagggucagcuu

SEQ ID NO: 7 - RNA, 31 nt, Homo sapiens
gggagacaggcaccacacacacacacacuuu